(19) United States
(12) Patent Application Publication
(10) Pub. No.: US 2023/0138400 A1
(43) Pub. Date: May 4, 2023

(54) METHODS AND PHARMACEUTICAL COMPOSITIONS FOR REPROGRAMING IMMUNE ENVIRONMENT IN A SUBJECT IN NEED THEREOF

(71) Applicants: INSERM (INSTITUT NATIONAL DE LA SANTE ET DE LA RECHERCHE MEDICALE), Paris (FR); UNIVERSITE PAUL SABATIER TOULOUSE III, Toulouse (FR); AFFICHEM, Toulouse (FR)

(72) Inventors: Sandrine SILVENTE POIROT, Toulouse (FR); Marc POIROT, Toulouse (FR); Julie LEIGNADIER, Toulouse (FR); Philippe DE MEDINA, Toulouse (FR); Michel RECORD, Toulouse (FR)

(21) Appl. No.: 17/849,217

(22) Filed: Jun. 24, 2022

Related U.S. Application Data
(62) Division of application No. 16/335,538, filed on Mar. 21, 2019, now abandoned.

(30) Foreign Application Priority Data
Sep. 22, 2016 (EP) 16306214.4

(51) Int. Cl. A61K 31/58; A61P 35/00; A61K 39/00; A61K 39/39; A61K 45/06

(52) U.S. Cl. CPC A61K 31/58; A61P 35/00; A61K 39/0011; A61K 39/39; A61K 45/06; A61K 2039/5154; A61K 2039/55511; A61K 2039/55555; A61K 2039/57

(57) ABSTRACT

The present invention relates to methods and pharmaceutical compositions for reprograming immune environment in a subject in need thereof. The inventors demonstrated that DDA induces differentiation of tumor cells and stimulates the secretion and the production of modified exosomes with anti-tumor properties (DDA-exosomes) via a mechanism dependent of the expression of the LXRbeta in the parental cells. In particular, one object of the present invention relates to a method of promoting Th1 differentiation and functionality and CD8+ cytotoxicity in a subject in need thereof comprising administering to the subject a therapeutically effective amount of DDA or DDA-exosomes.

### FIELD OF THE INVENTION

The present invention relates to methods and pharmaceutical compositions for reprograming immune environment in a subject in need thereof.

### BACKGROUND OF THE INVENTION

Dendrogenin A is a cholesterol metabolite with tumour suppressing properties whose production is impaired during oncogenesis (de Medina P, Paillasse M R, Segala G, Voisin M, Mhamdi L, Dalenc F, Lacroix-Triki M, Filleron T, Pont F, Saati T A, Morisseau C, Hammock B D, Silvente-Poirot S, Poirot M. Dendrogenin A arises from cholesterol and histamine metabolism and shows cell differentiation and anti-tumour properties. Nat Commun. 2013; 4:1840). The discovery of DDA opens up new promising opportunities for cancer treatments and new routes to understand the aetiology of cancers. It was shown that DDA arises from the stereoselective enzymatic conjugation of 5,6α-epoxy-cholesterol with histamine. DDA is detected in normal tissues from several organs but not in cancer cells and its level is decreased in breast tumors from patients, evidencing a deregulation of DDA metabolism during carcinogenesis. DDA is also able to control the growth of tumor cells implanted in mice and improves animal survival. In particular, it was observed that DDA-mediated tumour differentiation is accompanied by an increased infiltration of CD3+ T lymphocytes and CD11c+ dendritic cells (de Medina P, Paillasse M R, Segala G, Voisin M, Mhamdi L, Dalenc F, Lacroix-Triki M, Filleron T, Pont F, Saati T A, Morisseau C, Hammock B D, Silvente-Poirot S, Poirot M. Dendrogenin A arises from cholesterol and histamine metabolism and shows cell differentiation and anti-tumour properties. Nat Commun. 2013; 4:1840).

### SUMMARY OF THE INVENTION

The present invention relates to methods and pharmaceutical compositions for reprograming immune environment in a subject in need thereof. In particular, the present invention is defined by the claims.

### DETAILED DESCRIPTION OF THE INVENTION

The inventors now demonstrate that DDA induces differentiation of tumor cells and stimulates the secretion and the production of modified exosomes with anti-tumor properties (DDA-exosomes) via a mechanism dependent of the expression of the LXRbeta in the parental cells. The inventors showed that DDA-exosomes can stimulate the maturation of human dendritic cells (mDC) that produce cytokines which stimulate the polarization of naive T lymphocytes toward a CD4Th1 phenotype. IFNg produces by CD4 Th1 cells will favor the activation and recruitment of CD8 LT and the increase in the expression of tumor antigens at the tumor surface via the MHC. The inventors also demonstrate that DDA stimulates differentiation of monocytes into functional dendritic cells and increases the percent of CD4Th1 lymphocytes as well as their capacity to produce INFg. Accordingly, DDA is particularly suitable for reprograming immune environment in a subject in need thereof, more particularly in a subject suffering from cancer.

Accordingly, one object of the present invention relates to a method of promoting Th1 differentiation and functionality in a subject in need thereof comprising administering to the subject a therapeutically effective amount of DDA.

One object of the present invention relates to a method of inhibiting Treg differentiation in a subject in need thereof comprising administering to the subject a therapeutically effective amount of DDA. The method is thus particularly suitable for inhibiting an immunosuppressive response in the subject.

One object of the present invention relates to a method of promoting maturation of dendritic cells in a subject in need thereof comprising administering to the subject a therapeutically effective amount of DDA.

As used herein, the term “Dendrogenin A” or “DDA” refers to the pharmaceutically active compound 5a-hydroxy-6b-[2-(1H-imidazol-4-yl)ethylamino]cholestan-3b-ol. Dendrogenin A is disclosed in WO03/89449 and de Medina et al (J. Med. Chem., 2009). Its structural formula is the following:

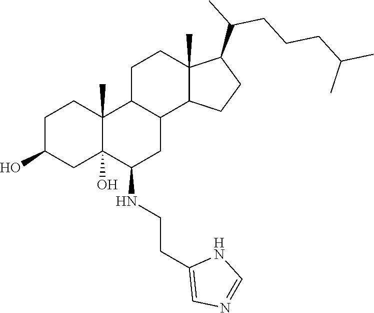 [SMILES: CC(C)CCCC(C)[C@H]1CCC2C3C[C@@H](NCCc4cnc[nH]4)[C@@]4(O)C[C@@H](O)CC[C@]4(C)C3CC[C@@]21C]

As used herein, the term “T helper cell” (“TH cell”) refers to a subset of lymphocytes which complete maturation in the thymus and have various roles in the immune system, including the identification of specific foreign antigens in the body and the activation and deactivation of other immune cells. By this, T helper cells are involved in almost all adaptive immune responses. Mature TH cells are believed to always express the surface protein CD4 and are therefore also termed CD4+ T cells. As used herein, the term “Th1 cell” and “Th2 cell” mean a type-1 helper T cell and a type-2 helper T cell, respectively. For instance Th1 cells produce high levels of the proinflammatory cytokine IFNγ. Polarization in said T cell subset can be carried out by any conventional method well known in the art that typically consists in incubation the T cells with at least one cytokine (e.g. IL12 for Th1 cells).

As used herein, the term ‘Treg’ or ‘T regulatory cell’ denotes a T lymphocyte endowed with a given antigen specificity imprinted by the TCR it expresses and with regulatory properties defined by the ability to suppress the response of conventional T lymphocytes or other immune cells. Different types of Tregs exist and include, but are not limited to: inducible and thymic-derived Tregs, as characterized by different phenotypes such as CD4+CD25+/high, CD4+CD25+/highCD127−/low alone or in combination with additional markers that include, but are not limited to, FoxP3, neuropilin-1 (CD304), glucocorticoid-induced TNFR-related protein (GITR), cytotoxic T-lymphocyte-associated protein 4 (CTLA-4, CD152); T regulatory type 1 cells; T helper 3 cells.

The term “dendritic cell”, as used herein, refers to any member of a diverse population of morphologically similar cell types found in lymphoid or non-lymphoid tissues. Dendritic cells are a class of “professional” antigen presenting cells, and have a high capacity for sensitizing HLA-restricted T cells. Specifically, the dendritic cells include, for example, plasmacytoid dendritic cells, myeloid dendritic cells (generally used dendritic cells, including immature and mature dendritic cells), Langerhans cells (myeloid dendritic cells important as antigen-presenting cells in the skin), interdigitating cells (distributed in the lymph nodes and spleen T cell region, and believed to function in antigen presentation to T cells). All these DC populations are derived from bone marrow hematopoietic cells. Dendritic cells also include follicular dendritic cells, which are important as antigen-presenting cells for B cells, but who are not derived from bone marrow hematopoietic cells. Dendritic cells may be recognized by function, or by phenotype, particularly by cell surface phenotype. These cells are characterized by their distinctive morphology (having veil-like projections on the cell surface), intermediate to high levels of surface HLA-class II expression and ability to present antigen to T cells, particularly to naive T cells. See Steinman R, et al., Ann. Rev. Immunol. 1991; 9:271-196. The cell surface of dendritic cells is characterized by the expression of the cell surface markers CD1a+, CD4+, CD86+, or HLA-DR+. The term “mature dendritic cell”, as used herein, is a cell that has significantly strong antigen-presenting ability for T cell or the like as compared with a dendritic cell in the immature state. Specifically, the mature dendritic cells may have an antigen-presenting ability that is half or stronger, preferably equivalent to or stronger than the antigen-presenting ability of dendritic cells in which maturation has been induced by adding LPS (1 μg/mL) and culturing for two days. Mature DC display up-regulated expression of co-stimulatory cell surface molecules and secrete various cytokines. Specifically, mature DCs express higher levels of HLA class I and class II antigens (HLA-A, B, C, HLA-DR) and are generally positive for the expression of CD80, CD83 and CD 86 surface markers. The expression “median tissue culture infective dose” or “TCID50”, as used herein, means the amount of a pathogenic agent that will produce pathological change in 50% of cell cultures inoculated.

The methods of the present invention are thus particularly suitable for the treatment of cancer.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to, cancer cells from the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In particular, DDA is administered to the patient for enhancing the proliferation, migration, persistence and/or cytoxic activity of CD8+ T cells in the subject and in particular the tumor-infiltrating of CD8+ T cells of the subject. As used herein “CD8+ T cells” has its general meaning in the art and refers to a subset of T cells which express CD8 on their surface. They are MHC class I-restricted, and function as cytotoxic T cells. “CD8+ T cells” are also called cytotoxic T lymphocytes (CTL), T-killer cells, cytolytic T cells, or killer T cells. CD8 antigens are members of the immunoglobulin supergene family and are associative recognition elements in major histocompatibility complex class I-restricted interactions.

Accordingly, the methods of the present invention are particularly suitable for the treatment of cancer characterized by a low tumor infiltration of CD8+ T cells. Accordingly a further object of the present invention relates to a method of treating cancer in a subject in need thereof comprising i) quantifying the density of CD8+ T cells in a tumor tissue sample obtained from the subject ii) comparing the density quantified at step i) with a predetermined reference value and iii) administering to the subject a therapeutically effective amount of DDA when the density quantified at step i) is lower than the predetermined reference value.

Typically said tumor-infiltration of CD8+ T cells is determined by any convention method in the art. For example, said determination comprises quantifying the density of CD8+ T cells in a tumor sample obtained from the subject. As used herein, the term “tumor tissue sample” means any tissue tumor sample derived from the patient. In some embodiments, the tumor tissue sample encompasses (i) a global primary tumor (as a whole), (ii) a tissue sample from the center of the tumor, (iii) a tissue sample from the tissue directly surrounding the tumor which tissue may be more specifically named the “invasive margin” of the tumor, (iv) lymphoid islets in close proximity with the tumor, (v) the lymph nodes located at the closest proximity of the tumor, (vi) a tumor tissue sample collected prior surgery (for follow-up of patients after treatment for example), and (vii) a distant metastasis. As used herein the “invasive margin” has its general meaning in the art and refers to the cellular environment surrounding the tumor. In some embodiments, the tumor sample may result from the tumor resected from the patient. In some embodiments, the tumor sample may result from a biopsy performed in the primary tumor of the patient or performed in metastatic sample distant from the primary tumor of the patient. The tumor tissue sample can, of course, be subjected to a variety of well-known post-collection preparative and storage techniques (e.g., fixation, storage, freezing, etc.). The sample can be fresh, frozen, fixed (e.g., formalin fixed), or embedded (e.g., paraffin embedded). In some embodiments, the quantification of density of CD8+ T cells is determined by immunohistochemistry (IHC). For example, the quantification of the density of CD8+ T cells is performed by contacting the tissue tumor tissue sample with a binding partner (e.g. an antibody) specific for a cell surface marker of said cells. Typically, the quantification of density of CD8+ T cells is performed by contacting the tissue tumor tissue sample with a binding partner (e.g. an antibody) specific for CD8. Typically, the density of CD8+ T cells is expressed as the number of these cells that are counted per one unit of surface area of tissue sample, e.g. as the number of cells that are counted per cm² or mm² of surface area of tumor tissue sample. In some embodiments, the density of cells may also be expressed as the number of cells per one volume unit of sample, e.g. as the number of cells per cm3 of tumor tissue sample. In some embodiments, the density of cells may also consist of the percentage of the specific cells per total cells (set at 100%). In some embodiments, the cell density of CD8+ T cells is determined in the whole tumor tissue sample, is determined in the invasive margin or center of the tumor tissue sample or is determined both in the centre and the invasive margin of the tumor tissue sample.

In some embodiments, the predetermined reference value correlates with the survival time of the subject. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”. In some embodiments, the predetermined value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical patient samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the density of CD8+ T cells in a group of reference, one can use algorithmic analysis for the statistic treatment of the measured densities in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

A further object of the present invention relates to a method for enhancing the potency of an immune checkpoint inhibitor administered to a patient as part of a treatment regimen, the method comprising administering to the patient a pharmaceutically effective amount of DDA in combination with the immune checkpoint inhibitor.

A further object of the present invention relates to a method of treating cancer in a patient in need thereof comprising administering to the patient a therapeutically effective combination of an immune checkpoint inhibitor with DDA, wherein administration of the combination results in enhanced therapeutic efficacy relative to the administration of the immune checkpoint inhibitor alone.

As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because the tumor microenvironment has relatively high levels of adenosine, which lead to a negative immune feedback loop through the activation of A2AR. B7-H3, also called CD276, was originally understood to be a co-stimulatory molecule but is now regarded as co-inhibitory. B7-H4, also called VTCN1, is expressed by tumor cells and tumor-associated macrophages and plays a role in tumor escape. B and T Lymphocyte Attenuator (BTLA), also called CD272, is a ligand of HVEM (Herpesvirus Entry Mediator). Cell surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152 is overexpressed on Treg cells serves to control T cell proliferation. IDO, Indoleamine 2,3-dioxygenase, is a tryptophan catabolic enzyme, a related immune-inhibitory enzymes. Another important molecule is TDO, tryptophan 2,3-dioxygenase. IDO is known to suppress T and NK cells, generate and activate Tregs and myeloid-derived suppressor cells, and promote tumor angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA. Short for V-domain Ig suppressor of T cell activation, VISTA is primarily expressed on hematopoietic cells so that consistent expression of VISTA on leukocytes within tumors may allow VISTA blockade to be effective across a broad range of solid tumors. As used herein, the term “PD-1” has its general meaning in the art and refers to programmed cell death protein 1 (also known as CD279). PD-1 acts as an immune checkpoint, which upon binding of one of its ligands, PD-L1 or PD-L2, inhibits the activation of T cells.

As used herein, the term “immune checkpoint inhibitor” has its general meaning in the art and refers to any compound inhibiting the function of an immune inhibitory checkpoint protein. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. In particular, the immune checkpoint inhibitor of the present invention is administered for enhancing the proliferation, migration, persistence and/or cytoxic activity of CD8+ T cells in the patient and in particular the tumor-infiltrating of CD8+ T cells of the patient. The ability of the immune checkpoint inhibitor to enhance T CD8 cell killing activity may be determined by any assay well known in the art. Typically said assay is an in vitro assay wherein CD8+ T cells are brought into contact with target cells (e.g. target cells that are recognized and/or lysed by CD8+ T cells). For example, the immune checkpoint inhibitor of the present invention can be selected for the ability to increase specific lysis by CD8+ T cells by more than about 20%, preferably with at least about 30%, at least about 40%, at least about 50%, or more of the specific lysis obtained at the same effector:target cell ratio with CD8+ T cells or CD8 T cell lines that are contacted by the immune checkpoint inhibitor of the present invention, Examples of protocols for classical cytotoxicity assays are conventional. Thus the expression “enhancing the potency of an immune checkpoint” refers to the ability of the DDA to increase the ability of the immune checkpoint inhibitor to enhance the proliferation, migration, persistence and/or cytoxic activity of CD8+ T cells.

In some embodiments, the immune checkpoint inhibitor is an antibody selected from the group consisting of anti-CTLA4 antibodies, anti-PD-1 antibodies, anti-PD-L1 antibodies, anti-PD-L2 antibodies anti-TIM-3 antibodies, anti-LAG3 antibodies, anti-B7H3 antibodies, anti-B7H4 antibodies, anti-BTLA antibodies, and anti-B7H6 antibodies.

Examples of anti-CTLA-4 antibodies are described in U.S. Pat. Nos. 5,811,097; 5,811,097; 5,855,887; 6,051,227; 6,207,157; 6,682,736; 6,984,720; and 7,605,238. One anti-CTLA-4 antibody is tremelimumab, (ticilimumab, CP-675,206). In some embodiments, the anti-CTLA-4 antibody is ipilimumab (also known as 10D1, MDX-D010) a fully human monoclonal IgG antibody that binds to CTLA-4.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9 (Gal9). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011155607, WO2013006490 and WO2010117057.

In some embodiments, the immune checkpoint inhibitor is an IDO inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In some embodiments, the immune checkpoint inhibitor is a PD-1 inhibitor. Accordingly, the term “PD-1 inhibitor” as used herein refers to a compound, substance or composition that can inhibit the function of PD-1. For example, the inhibitor can inhibit the expression or activity of PD-1, modulate or block the PD-1 signaling pathway and/or block the binding of PD-1 to PD-L1 or PD-L2.

In some embodiments, the PD-1 inhibitor is an antibody directed against the extracellular domain of PD-1. In some embodiments, the PD-1 inhibitor is an antibody directed against the extracellular domain of PD-L1. Examples of PD-1 and PD-L1 antibodies are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In some embodiments, the PD-1 blockers include anti-PD-L1 antibodies. In certain other embodiments the PD-1 blockers include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224 is a fusion protein of B7-DC; an antibody Fc portion; BMS-936559 (MDX-1105-01) for PD-L1 (B7-H11) blockade.

In some embodiments, the PD-1 inhibitor is a small molecule or peptide, or a peptide derivative, such as those described in U.S. Pat. Nos. 8,907,053; 9,096,642; and 9,044,442 and U S Patent Application Publication No 2015/0087581; 1,2,4 oxadiazole compounds and derivatives such as those described in U.S. Patent Application Publication No. 2015/0073024; cyclic peptidomimetic compounds and derivatives such as those described in U.S. Patent Application Publication No. 2015/0073042; cyclic compounds and derivatives such as those described in U.S. Patent Application Publication No. 2015/0125491; 1,3,4 oxadiazole and 1,3,4 thiadiazole compounds and derivatives such as those described in International Patent Application Publication No. WO 2015/033301; peptide-based compounds and derivatives such as those described in International Patent Application Publication Nos WO 2015/036927 and WO 2015/04490, or a macrocyclic peptide-based compounds and derivatives such as those described in U.S. Patent Application Publication No 2014/0294898; the disclosures of each of which are hereby incorporated by reference in their entireties.

As used herein the term “co-administering” as used herein means a process whereby the combination of the DDA and the immune checkpoint inhibitor, is administered to the same patient. The DDA and the immune checkpoint inhibitor may be administered simultaneously, at essentially the same time, or sequentially. If administration takes place sequentially, the DDA is administered before the immune checkpoint inhibitor. The DDA and the immune checkpoint inhibitor need not be administered by means of the same vehicle. The DDA and the immune checkpoint inhibitor may be administered one or more times and the number of administrations of each component of the combination may be the same or different. In addition, the SK1 inhibitor and the immune checkpoint inhibitor need not be administered at the same site.

As used herein, the expression “enhanced therapeutic efficacy,” relative to cancer refers to a slowing or diminution of the growth of cancer cells or a solid tumor, or a reduction in the total number of cancer cells or total tumor burden. An “improved therapeutic outcome” or “enhanced therapeutic efficacy” therefore means there is an improvement in the condition of the patient according to any clinically acceptable criteria, including, for example, decreased tumor size, an increase in time to tumor progression, increased progression-free survival, increased overall survival time, an increase in life expectancy, or an improvement in quality of life. In particular, “improved” or “enhanced” refers to an improvement or enhancement of 1%, 5%, 10%, 25% 50%, 75%, 100%, or greater than 100% of any clinically acceptable indicator of therapeutic outcome or efficacy. As used herein, the expression “relative to” when used in the context of comparing the activity and/or efficacy of a combination composition comprising the immune checkpoint inhibitor with the DDA to the activity and/or efficacy of the immune checkpoint inhibitor alone, refers to a comparison using amounts known to be comparable according to one of skill in the art.

The inventors also demonstrate that the immune effects induced by the administration of DDA depends on the expression of LXRβ. Accordingly a further object of the present invention relates to a method for the treatment of cancer characterized by a the expression of LXRβ. Accordingly a further object of the present invention relates to a method of treating cancer in a subject in need thereof comprising i) quantifying the expression level of LXRβ in a tumor tissue sample obtained from the subject ii) comparing expression level determined at step i) with a predetermined reference value and iii) administering to the subject a therapeutically effective amount of DDA when the expression level quantified at step i) is higher than the predetermined reference value.

As used herein, the term LXRβ refers to liver X receptor beta, also named Oxysterols receptor LXR-beta (amino acid sequence Uniprot reference: P55055), which is a member of the nuclear receptor family of transcription factors. LXRβ is encoded by the LXRβ gene (nucleic acids sequence NCBI Gene ID: 7376).

In some embodiments, the expression of LXRβ is determined at the protein level by, any well know method in the art such as e.g. any immunoassays well known in the art. For instance, the expression level of LXRβ may be determined by immunohistochemistry. Immunohistochemistry typically includes the following steps i) fixing the tumor tissue sample with formalin, ii) embedding said tumor tissue sample in paraffin, iii) cutting said tumor tissue sample into sections for staining, iv) incubating said sections with the binding partner specific for LXRβ, v) rinsing said sections, vi) incubating said section with a secondary antibody typically biotinylated and vii) revealing the antigen-antibody complex typically with avidin-biotin-peroxidase complex. Accordingly, the tumor tissue sample is firstly incubated with the binding partners having for LXRβ. After washing, the labeled antibodies that are bound to SMAase2 are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. Hematoxylin & Eosin, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein. Exemplary labels include radioactive isotopes, fluorophores, ligands, chemiluminescent agents, enzymes, and combinations thereof. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. ³H, ¹⁴C, ³²P, ³⁵S or ¹²⁵I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In some embodiments, the label is a quantum dot. For example, Quantum dots (Qdots) are becoming increasingly useful in a growing list of applications including immunohistochemistry, flow cytometry, and plate-based assays, and may therefore be used in conjunction with this invention. Qdot nanocrystals have unique optical properties including an extremely bright signal for sensitivity and quantitation; high photostability for imaging and analysis. A single excitation source is needed, and a growing range of conjugates makes them useful in a wide range of cell-based applications. Qdot Bioconjugates are characterized by quantum yields comparable to the brightest traditional dyes available. Additionally, these quantum dot-based fluorophores absorb 10-1000 times more light than traditional dyes. The emission from the underlying Qdot quantum dots is narrow and symmetric which means overlap with other colors is minimized, resulting in minimal bleed through into adjacent detection channels and attenuated crosstalk, in spite of the fact that many more colors can be used simultaneously. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. In some embodiments, the resulting stained specimens are each imaged using a system for viewing the detectable signal and acquiring an image, such as a digital image of the staining. Methods for image acquisition are well known to one of skill in the art. In some embodiments, it is advantageous for the technique to preserve the localization of the biomarker and be capable of distinguishing the presence of biomarkers in cancerous and non-cancerous cells. Such methods include layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 2011/0306514 (incorporated herein by reference); and in Chung & Hewitt, Meth Mol Biol, Prot Blotting Detect, Kurlen & Scofield, eds. 536: 139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, Md.). In some embodiments, the L-IHC method can be performed on any of a variety of tissue samples, whether fresh or preserved. The samples included core needle biopsies that were routinely fixed in 10% normal buffered formalin and processed in the pathology department. Standard five ptu thick tissue sections were cut from the tissue blocks onto charged slides that were used for L-IHC. Thus, L-IHC enables testing of multiple markers in a tissue section by obtaining copies of molecules transferred from the tissue section to plural bioaffinity-coated membranes to essentially produce copies of tissue “images.” In the case of a paraffin section, the tissue section is deparaffinized as known in the art, for example, exposing the section to xylene or a xylene substitute such as NEO-CLEAR®, and graded ethanol solutions. The section can be treated with a proteinase, such as, papain, trypsin, proteinase K and the like. Then, a stack of a membrane substrate comprising, for example, plural sheets of a 10μιη thick coated polymer backbone with 0.4μιη diameter pores to channel tissue molecules, such as, proteins, through the stack, then is placed on the tissue section. The movement of fluid and tissue molecules is configured to be essentially perpendicular to the membrane surface. The sandwich of the section, membranes, spacer papers, absorbent papers, weight and so on can be exposed to heat to facilitate movement of molecules from the tissue into the membrane stack. A portion of the proteins of the tissue are captured on each of the bioaffinity-coated membranes of the stack (available from 20/20 GeneSystems, Inc., Rockville, Md.). Thus, each membrane comprises a copy of the tissue and can be probed for a different biomarker using standard immunoblotting techniques, which enables open-ended expansion of a marker profile as performed on a single tissue section. As the amount of protein can be lower on membranes more distal in the stack from the tissue, which can arise, for example, on different amounts of molecules in the tissue sample, different mobility of molecules released from the tissue sample, different binding affinity of the molecules to the membranes, length of transfer and so on, normalization of values, running controls, assessing transferred levels of tissue molecules and the like can be included in the procedure to correct for changes that occur within, between and among membranes and to enable a direct comparison of information within, between and among membranes. Hence, total protein can be determined per membrane using, for example, any means for quantifying protein, such as, biotinylating available molecules, such as, proteins, using a standard reagent and method, and then revealing the bound biotin by exposing the membrane to a labeled avidin or streptavidin; a protein stain, such as, Blot fastStain, Ponceau Red, brilliant blue stains and so on, as known in the art.

In some embodiments, the expression the expression of LXRβ is determined at the nucleic acid level (e.g. mRNA). For instance, methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In some embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook-A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antl1ranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. 10 Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof.

Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6, 130, 101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649,138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlsbad, Calif.). Additional labels include, for example, radioisotopes (such as 3H), metal chelates such as DOTA and DPTA chelates of radioactive or paramagnetic metal ions like Gd3+, and liposomes. Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase. Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pir1kel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 0.1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrazoles, thiazoles, nitroaryls, benzofurazans, triterpenes, ureas, thioureas, rotenones, coumarin, coumarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In some embodiments, the methods of the invention comprise the steps of providing total RNAs extracted from cumulus cells and subjecting the RNAs to amplification and hybridization to specific probes, more particularly by means of a quantitative or semi-quantitative RT-PCR.

In some embodiments, the level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In some embodiments, the nCounter® Analysis system is used to detect intrinsic gene expression. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tumor tissue sample with a probe library, such that the presence of the target in the sample creates a probe pair-target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Expression level of a gene may be expressed as absolute level or normalized level. Typically, levels are normalized by correcting the absolute level of a gene by comparing its expression to the expression of a gene that is not a relevant for determining the cancer stage of the subject, e.g., a housekeeping gene that is constitutively expressed. Suitable genes for normalization include housekeeping genes such as the actin gene ACTB, ribosomal 18S gene, GUSB, PGK1 and TFRC. This normalization allows the comparison of the level in one sample, e.g., a subject sample, to another sample, or between samples from different sources.

In some embodiments, expression level of LXRβ quantified at step i) is compared to a predetermined reference value, which is a threshold value or a cut-off value. As explained above, the threshold value can also be arbitrarily selected based upon the existing experimental and/or clinical conditions, as would be recognized by a person of ordinary skilled in the art. For example, retrospective measurement of expression level of the gene in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data.

In some embodiments, when the expression level of LXRβ quantified at step i) is higher than the predetermined reference value, DDA is administered to the patient in combination with an immune checkpoint inhibitor as above described.

One further object of the present invention relates to a vaccine composition comprising an immunoadjuvant together with one or more antigens, for inducing an immune response against said one or more antigens wherein the immunoadjuvant is DDA.

As used herein, the term “vaccine composition” has its general meaning in the art and refers to a composition that can be administered to humans or to animals in order to induce an immune system response; this immune system response can result in a production of antibodies or simply in the activation of certain cells, in particular antigen-presenting cells, T lymphocytes (in particular T-CD8+ cells) and B lymphocytes. The vaccine composition can be a composition for prophylactic purposes or for therapeutic purposes or both. In particular, the vaccine composition of the present invention is used to protect healthy individuals from developing tumors with known antigenic components (“tumor protective vaccine”). In such a case the patient would be treated with known tumor antigens or his own (excised) tumor material targeted in such a fashion to the myeloid dendritic cell of the invention, as to elicit a powerful cytotoxic Th1 immune response against tumor specific antigens.

As used herein, the term “immunoadjuvant” refers to a compound that can induce and/or enhance the immune response against an antigen when administered to a subject or an animal. It is also intended to mean a substance that acts generally to accelerate, prolong, or enhance the quality of specific immune responses to a specific antigen. In the context of the present invention, the term “immunoadjuvant” means a compound, which enhances both innate immune response by affecting the transient reaction of the innate immune response and the more long-lived effects of the adaptive immune response by activation and maturation of the antigen-presenting cells (APCs) especially Dendritic cells (DCs).

As used herein the term “antigen” refers to a molecule capable of being specifically bound by an antibody or by a T cell receptor (TCR) if processed and presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. An antigen can have one or more epitopes or antigenic sites (B- and T-epitopes).

A variety of substances can be used as antigens in a compound or formulation, of immunogenic or vaccine type. For example, attenuated and inactivated viral and bacterial pathogens, purified macromolecules, polysaccharides, toxoids, recombinant antigens, organisms containing a foreign gene from a pathogen, synthetic peptides, polynucleic acids, antibodies and tumor cells can be used to prepare the vaccine composition of the present invention. Therefore, the immunoadjuvant of the present invention (i.e. DDA) can be combined with a wide variety of antigens to produce a vaccine composition useful for inducing an immune response in a subject. Those skilled in the art will be able to select an antigen appropriate for treating a particular pathological condition and will know how to determine whether an isolated antigen is favored in a particular vaccine formulation.

In some embodiments, the antigen is a protein or peptide coded by a DNA or other suitable nucleic acid sequence which has been introduced in cells by transfection, lentiviral or retroviral transduction, mini-gene transfer or other suitable procedures. In some embodiments, said antigen is a protein which can be obtained by recombinant DNA technology or by purification from different tissue or cell sources. Typically, said protein has a length higher than 10 amino acids, preferably higher than 15 amino acids, even more preferably higher than 20 amino acids with no theoretical upper limit. Such proteins are not limited to natural ones, but also include modified proteins or chimeric constructs, obtained for example by changing selected amino acid sequences or by fusing portions of different proteins. In some embodiments, said antigen is a synthetic peptide. Typically, said synthetic peptide is 3-40 amino acid-long, preferably 5-30 amino acid-long, even more preferably 8-20 amino acid-long. Synthetic peptides can be obtained by Fmoc biochemical procedures, large-scale multipin peptide synthesis, recombinant DNA technology or other suitable procedures. Such peptides are not limited to natural ones, but also include modified peptides, post-translationally modified peptides or chimeric peptides, obtained for example by changing or modifying selected amino acid sequences or by fusing portions of different proteins.

In some embodiments, the antigen is a viral antigen. Examples of viral Ags include but are not limited to influenza viral Ags (e.g. hemagglutinin (HA) protein, matrix 2 (M2) protein, neuraminidase), respiratory syncitial virus (RSV) Ags (e.g. fusion protein, attachment glycoprotein), polio, papillomaviral (e.g. human papilloma virus (HPV), such as an E6 protein, E7 protein, L1 protein and L2 protein), Herpes simplex, rabies virus and flavivirus viral Ags (e.g. Dengue viral Ags, West Nile viral Ags), hepatitis viral Ags including Ags from HBV and HCV, human immunodeficiency virus (HIV) Ags (e.g. gag, pol or nef), herpesvirus (such as cytomegalovirus and Epstein-Barr virus) Ags (e.g. pp65, IE1, EBNA-1, BZLF-1) and adenovirus Ags.

In some embodiments, the antigen is a bacterial antigen. Examples of bacterial Ags include but are not limited to those from Streptococcus pneumonia, Haemophilus influenza, Staphylococcus aureus, Clostridium difficile and enteric gram-negative pathogens including Escherichia, Salmonella, Shigella, Yersinia, Klebsiella, Pseudomonas, Enterobacter, Serratia, Proteus, B. anthracis, C. tetani, B. pertussis, S. pyogenes, S. aureus, N. meningitidis and Haemophilus influenzae type b.

In some embodiments, the antigen is a fungal or protozoal antigen. Examples include but are not limited to those from Candida spp., Aspergillus spp., Crytococcus neoformans, Coccidiodes spp., Histoplasma capsulatum, Pneumocystis carinii, Paracoccidioides brasiliensis, Plasmodium falciparum, Plasmodium vivax, Plasmodium ovale, and Plasmodium malariae.

In some embodiments, the antigen of the vaccine composition is a “Tumor associated antigen” or “TAA”. As used herein, the term “tumor associated antigen” refers to an antigen that is characteristic of a tumor tissue. Examples of TAAs include, without limitation, CEA, prostate specific antigen (PSA), HER-2/neu, BAGE, GAGE, MAGE 1-4, 6 and 12, MUC-related protein (Mucin) (MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, prostate serum antigen (PSA), PRAME (melanoma antigen), β-catenin, MUM-1-B (melanoma ubiquitous mutated gene product), GAGE (melanoma antigen) 1, BAGE (melanoma antigen) 2-10, C-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67. In some embodiments, the antigen is selected from tumor associated antigens comprising antigens from leukemias and lymphomas, neurological tumors such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

In some embodiments, the vaccine composition comprises at least one population of antigen presenting cells that present the selected antigen. The antigen-presenting cell (or stimulator cell) typically has an MHC class I or II molecule on its surface, and in one embodiment is substantially incapable of itself loading the MHC class I or II molecule with the selected antigen. Preferably, the antigen presenting cells are dendritic cells. Suitably, the dendritic cells are autologous dendritic cells that are pulsed with the antigen of interest (e;g. a peptide). T-cell therapy using autologous dendritic cells pulsed with peptides from a tumor associated antigen is disclosed in Murphy et al. (1996) The Prostate 29, 371-380 and Tjua et al. (1997) The Prostate 32, 272-278. Thus, in some embodiments, the vaccine composition containing at least one antigen presenting cell is pulsed or loaded with one or more antigenic peptides. As an alternative the antigen presenting cell comprises an expression construct encoding an antigenic peptide. The polynucleotide may be any suitable polynucleotide and it is preferred that it is capable of transducing the dendritic cell, thus resulting in the presentation of a peptide and induction of an immune response.

A further aspect of the invention relates to a method for vaccinating a subject in need thereof comprising administering a pharmaceutically effective amount of the vaccine composition of the present invention. In particular, the vaccine composition of the present invention is particularly suitable for the treatment of cancer in a subject in need thereof.

One further object of the present invention relates to a method of generating a population of exosomes (DDA-exosomes) comprising contacting a population of tumor cells with an amount of DDA for a time sufficient to induce exosomes releasing by the population of tumor cells.

As used herein, the term “exosome” has its general meaning in the art and refers to a nanometer-sized (30 nm to 150 nm, e.g., 40 nm to 100 nm) vesicle that originates as an internal vesicle of a multivesicular body (MVB), present in endocytic and secretory pathways. Exosomes are formed by an invagination process or inward budding which causes a membrane-enclosed compartment in which the lumen is topologically equivalent of cytoplasm. In particular the exosomes produced by the method of the present invention are tumor exosomes. It should be understood that the term “tumor exosome” includes both intact tumor exosomes and fragmented tumor exosomes.

Typically, the population of tumor cells is contacted with an effective amount of DDA for a time ranging from 24 to 48 hours.

The tumor exosomes obtainable by the method of the present invention are particularly suitable for preparing vaccine compositions. Thus a further object of the present invention relates to a vaccine composition comprising an immunoadjuvant together with one or more antigens, for inducing an immune response against said one or more antigens wherein the immunoadjuvant is a population of tumor exosomes of the present invention.

The tumor exosomes obtainable by the method of the present invention are also particularly suitable for the treatment of cancer. Accordingly, one further object of the present invention relates to a method of treating cancer in a subject in need thereof comprising administering to the subject a therapeutically effective amount of the tumor exosomes of the present invention.

Typically the subject is administered with a composition enriched for the exosomes produced at step i). For example centrifugation and/or chromatography, such as size-exclusion chromatography can be used for enriching the composition for exosomes. For example, an enriched composition comprises at least 10% of the desired component (e.g., exosomes); in other embodiments, the enriched sample comprises at least 20%, at least 30%, at least 40%, at least 50%, at least 60%, at least 70%, at least 80%, at least 90%, at least 95%, or at least 99% of the desired component. Thus, an exosome-enriched composition refers to a composition that comprises at least 10% exosomes as determined by, e.g., measuring the level of an exosome cell surface antigen such as those described in e.g., U.S. Pat. No. 7,198,923. In some embodiments, an exosome-enriched composition comprises e.g., at least 20%, at least 30%, at least 40%, at least 50%, at least 60%, at least 70%, at least 80%, at least 90%, at least 95%, or at least 99% exosomes. In some embodiments, the exosomes expressing a particular antigen which produced at step i) are previously purified before being administered to the subject. The purification of exosomes can be accomplished, for example, by using antibodies, aptamers, aptamer analogs or molecularly imprinted polymers specific for a desired surface antigen. In one embodiment, the surface antigen is specific for a cancer type. One example of a method of exosome separation based on cell surface antigen is provided in U.S. Pat. No. 7,198,923. As described in, e.g., U.S. Pat. Nos. 5,840,867 and 5,582,981, and WO/2003/050290, aptamers and their analogs specifically bind surface molecules and can be used as a separation tool for retrieving cell type-specific exosomes.

A further object of the present invention relates to a method of treating cancer in a subject in need thereof comprising i) quantifying the expression level of LXRβ in a tumor tissue sample obtained from the subject ii) comparing expression level determined at step i) with a predetermined reference value and iii) administering to the subject a therapeutically effective amount of DDA-exosomes when the expression level quantified at step i) is lower than the predetermined reference value. The expression of LXRβ is determined as described above.

By a “therapeutically effective amount” is meant a sufficient amount of compound or composition at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The composition of the present invention typically comprises pharmaceutically acceptable excipients, and optionally sustained-release matrices, such as biodegradable polymers, to be administered in the form of a pharmaceutical composition. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The compound can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active antibody in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIGS. 1A-E. Sorted naive CD4 T cells were isolated from the spleen of C57BL/6 mice and activated in the indicated Th1, Th2, Treg, or Th17 polarizing conditions as described in the “Materials and methods” section”. These cells were cultured in the presence of increasing concentrations of DDA. After 96 hours, the percentage of (A) Th1: CD4+Tbet+IFNg+, (B) Th2: CD4+GATA3+IL6+, (C) Treg: CD4+Foxp3+IL10+ and (D) Th17: CD4+RORgt+IL6+, cells was measured by flow cytometry. (E) Sorted naive CD8 T cells were isolated from the spleen of the C57BL/6 mice and activated with anti-CD3, anti-CD28 and recombinant IL2. After 96 hours, the percentage of cytotoxic T CD8+GranzymB+IFNg+ cells was measured by flow cytometry.

FIGS. 2A-E. Sorted naive CD4 T cells isolated from the spleen of C57BL/6 mice were activated in Th1, Th2, Treg, or Th17 polarizing conditions as indicated in the “Materials and methods” section”. After 96 h, increasing concentrations of DDA was added on polarized CD4 T cells for 24 h. Then the percentage of (A) CD4+Tbet+IFNg+, (B) CD4+GATA3+IL6+, (C) CD4+Foxp3+IL10+ and (D) CD4+RORgt+IL6+ cells was measured by flow cytometry. (E) Sorted naïve CD8 T cells were isolated from the spleen of the C57BL/6 mice and activated with anti-CD3, anti-CD28 and recombinant IL2. After 96 h, increasing concentrations of DDA was added on activated CD8 T cells for 24 h. Then the percentage of cytotoxic T CD8+GranzymB+IFNg+ cells was measured by flow cytometry.

FIGS. 3A-D. Unpolarized Th0 (prepared from naive CD4 T cells isolated from the spleen of C57BL/6 mice were cultivated in presence of anti-CD3, anti-CD28 and recombinant IL-2 as indicated in the “Materials and methods” section”) and with gradual concentration of DDA added at day 0 of culture (condition #1) or Day 4 of culture (condition #2). At the end, for each condition, the percentage of Th1 (CD4+Tbet+IFNg+) (A,B) and Treg CD4+Foxp3+IL10+ (C,D) was measured by flow cytometry.

FIGS. 4A-C. Tumor growth analysis (A) Exponentially growing E0771 cells were collected, washed twice in PBS and resuspended in PBS (300,000 cells in 100 μl PBS). E0771 tumors were prepared by subcutaneous transplantation into the flanks of C57BL/6 mice. When tumor measured 50 mm3, the mice were treated every 5 days with 0.37 μg/kg or 20 mg/kg DDA or with the solvent vehicle (control). (B) The tumor volume was determined by direct measurement with a caliper and was calculated using the formula (width2×length)/2. (C) The Kaplan-Meier method was used to compare the percentage of animal with tumor<2000 mm3.

FIGS. 5A-D. Infiltration of immune cells inside E0771 tumor. (A-D) These bar graphs represent the ratio between (A) Th1 (CD4+ Tbet+) and Treg (CD4+ Foxp3+), (B) CTL cells (CD8+ Granzym B+) on No CTL cells (CD8+ Granzym b−), (C) macrophage type M1 (CD14+ CCR7+ IFNg+) and type M2 (CD14+ CD206+ IL10+) (D) dendritic cells (CD11c+) and myeloid derived suppressive cells (MDSC: CD11b+CD11clow, LY6C+ly6Gint) infiltrated inside the tumor. The tumors were removed at day 15 post treatment with DDA at 0.37 μg/kg (b) or with the solvent vehicle (control) (a). To observe the cytotoxic CD8 T cells, the tumor suspension was prealably stimulated 2 h in vitro with a cocktail of PMA (50 ng/ml), ionomycine (500 ng/ml) and golgistop (concentration from manufacture BD Pharmagen), then the cells were stained with specific antibodies.

FIGS. 6A-B. The analysis of exosomes secreted from B16F10 cells after DDA (called DDA-exosomes) or the solvent vehicle (called control-exosomes) treatments demonstrates that DDA modifies their composition. (A) DDA-exosomes are enriched in proteins with antigen-presentating properties such as CD1d, MHC-II and Hsp70, with differentiation antigenes such as tyrosinase. (B) DDA-exosomes present also a decrease level in PGE2, an immunosuppressive lipid, compared with control-exosomes. DDA-exosomes may thus activate the immune system against the tumor. It has to be noted that the exosomes produced from tumor cells are described in the literature as immuno-suppressor and pro-tumor. This is due to the fact that tumor exosomes are enriched in immuno-suppressive molecules, such as PGE2.

FIG. 7. A single intra-dermal injection of DDA-exosomes (1 μg/mouse) purified from the media of B16F10 cells treated with 1 μM DDA for 24 h into the flank of mice grafted with a B16F10 tumor, inhibits tumor growth and increases mice survival compared with injection of control-exosomes in same conditions. DDA-exosomes inhibit tumor growth and increase mice survival. DDA is the first molecule, to our knowledge, to be able to stimulate the production of anti-tumor exosomes from tumor cells. We have a pharmacological modification of the phenotype and activity of tumor exosomes by DDA.

FIG. 8. DDA-exosomes purified from human SKMEL-28-shCTRL cells media increase cell surface markers of mature human dendritic cells.

FIGS. 9A-B. Sorted naive CD4 T cells were isolated from the spleen of WT or LXRαβKO mice (collaboration with Hervé Guillou, INRA, Toulouse) and activated in the indicated Th1 or Treg polarizing conditions. These cells were cultured in the presence of increasing concentrations of DDA. After 96 hours, the percentage of (A) Th1: CD4+Tbet+IFNg+ and (B) Treg: CD4+Foxp3+IL10+ cells was measured by flow cytometry and expressed relative to the control.

FIG. 10. Sorted naive CD4 T cells were isolated from the spleen of WT or LXRαβKO mice and activated in Th2 polarizing conditions. These cells were cultured in the presence of increasing concentrations of DDA. After 96 hours, the percentage of Th2: CD4+GATA3+IL4+ cells was measured by flow cytometry and expressed relative to the control.

FIGS. 11A-B. Bone marrow was isolated from the tibia and femur of WT or LXRαβKO mice and cultivated with 20 ng/ml GM-CSF and in presence of increasing concentrations of DDA. At day 3 and 5 half of medium was replaced by fresh medium containing GM-CSF. After 7 days of culture, the percentage of (A) differentiated CD11c+CD8a dendritic cell (B) mature CD11c+CD8a+ dendritic cells (CD11c+CD8a+CD86hi CCR7hi) was measured by flow cytometry and expressed relative to the control.

FIG. 12. Bone marrow was isolated from the tibia and femur of WT or LXRαβKO mice and cultivated with 20 ng/ml GM-CSF and in presence of increasing concentrations of DDA. At day 3 and 5 half of medium was replaced by fresh medium containing GM-CSF. After 7 days of culture, the mean of florescence (MFI) of MHC class II expressed on the surface of CD11c dendritic cells: CD11c+H2db+ was measured by flow cytometry and expressed relative to the control.

FIGS. 13A-B. Tumor growth analysis (A) Exponentially growing E0771 sh control or E0771 sh LXR cells were collected, washed twice in PBS and resuspended in PBS (300,000 cells in 100 μl PBS). C57BL/7 mice were grafted subcutaneously with 300 000 E0771 sh control or E0771 sh LXR tumor cells. When the tumors reached a volume of 50 mm³, the mice were treated every 2 days with 0.37 μg/kg DDA or treated with the solvent vehicle (untreated). (B) The tumor volume was determined by direct measurement with a caliper and was calculated using the formula (width2×length)/2. At day 15 of treatment with DDA or the solvent vehicle, the tumors were removed and the immune cells infiltrated into the tumors were analyzed by flow cytometry (see FIG. 14).

FIGS. 14A-D. C57BL/7 mice were grafted subcutaneously with 300 000 E0771 tumor cells knocked down for the LXR (E0771 sh LXR) or with control cells (E0771 Sh control). When the tumors reached a volume of 50 mm³, the mice were treated or not with 0.37 μg/kg of DDA every 2 days. 15 days post DDA treatment, the tumor cells were collected to assess by flow cytometry the CD4 Th1 cells (CD4+ Tbet+), CD4 T regulatory (Treg: CD4+ Foxp3+), CD8 T cells cytotoxic (CTL: CD8+ granzyme +)) or not (non CTL: CD8+ Granzyme −), macrophage M1 (F4/80+ CD206− CD86+) and M2 (F4/80+ CD206+ CD86−), dendritic cells CD11c+, CD11c+CD8α+ and myeloid derived suppressive cells (MDSC: CD11b+CD11clow, LY6C+ly6Gint) infiltrated into the tumors. To observe the cytotoxic CD8 T cells, the tumor suspension was prealably stimulated 2 h in vitro with a cocktail of PMA (50 ng/ml), ionomycine (500 ng/ml) and golgistop (concentration from manufacture BD Pharmagen), then the cells were stained with specific antibodies. The bars graphs represent the ratio between (A) Th1 and Treg, (B) CTL and non CTL, (C) M1 and M2 and (D) Alls DCs (CD11c+ and CD11c+ CD8α+) and MDSC infiltrated inside the tumors: E0771 sh control or E0771 Sh LXR (knocked down for LXRβ) from mice treated or not with DDA at 0.37 ug/kg. In cells expressing the LXRβ (E0771 shcontrol cells), DDA increases the infiltration of activated immune cells (Th1, CTL, macrophages M1 and DC) and decreases the infiltration of immunosuppressives cells (Treg, non CTL, M2 and MDSC). In cells knocked down for the LXRβ (E0771 shLXR cells), the effect of DDA is significantly decreased.

FIGS. 15A-D. DDA treatment decreases the percentage of T regulatory CD4 T cells and increases the activated CD4+ and CD8+ T cells inside tumors. Immunocompetent C57BL/6 mice (Janvier Laboratory) were implanted subcutaneously with 300 000 E0771 (ER+) mouse mammary tumor cells expressing the LXRb (wild type cells). When tumors were palpable (50 mm3), the animals were treated with the vehicle (empty symbol) or s.c. 0.37 μg/kg of DDA (full symbol) every 2 days, once a day. Fifty days post treatment, the animals were sacrified to collect the tumors. The tumors were dissociated by using gentlemac technology (Miltenyi) and then the suspension of tumor cells were stimulated with 50 ng/ml of PMA (Sigma), 500 ng/ml Ionomycin (Sigma) and 1/1000 of golgi stop (ebiosicence) during 3 h at 37° C. Tumor infiltrated Lymphocytes (TIL) isolated from E0711 tumors were stained with antibodies against CD45, CD8, CD4, PD-1, Foxp3, T-bet, IFN-g, Granzym B, PD-1 as well as live/dead stain. By gating on CD45+CD4+ (A-B) or CD45+CD8+ T cells populations (C-D), the tumor-infiltrating (A) T regulatory cells (Treg; Foxp3+), (B) effector CD4+ cells (Th1; T-bet+) and (C) cytotoxic CD8 T cells (CTL; IFN-γ+Granzym B+) were analyzed by flow cytometry. The vehicle condition is normalized at 1 and the graphs are representative of three independent experiments. (D). The graph represents the percentage of PD-1 negative CD8+ cells inside tumors of mice treated with vehicle or DDA and is representative of three independent experiments.

FIGS. 16A-C. The control of tumor growth and the increase in animal survival upon DDA treatment are dependent on the LXRβ expressed in tumor cells. Immunocompetent C57BL/6 mice (Janvier Laboratory) were implanted subcutaneously with 300 000 E0771 (ER+) mouse mammary tumor cells expressing the LXRb (E0 shC, square symbol) or knockdown for the LXRb expression with shRNA (shLXRb, circle symbol). When the tumor reached a volume of 50-100 mm3, animals (n=12 mice per group) were treated intraperitoneally once per day, every two days with 0.37 μg/kg of DDA (Affichem). The animals were monitored over time for (A) tumor growth and (B) animal survival. (C) Tumor weights were measured at the end of the experiment. The tumor volume was determined by direct measurement with a caliper and was calculated using the formula (width2×length)/2. The mean tumour volume±s.e.m is shown. The Kaplan-Meier method was used to compare mice survival.

FIGS. 17A-D. The silencing of LXRβ in tumor affects the effects of DDA on T regulatory and cytotoxic CD8+ T cells population infiltrated inside tumor. Animals grafted with E0711 tumors expressing the LXRβ (square symbol) or knockdown for the LXRβ expression (circle symbol) treated in FIG. 2 were sacrified to collect tumors, 15 days post treatment with the vehicle (empty symbol) or 0.37 μg/kg of DDA (full symbol). The tumor were dissociated by using gentlemac technology (Miltenyi), and the suspension of tumor cells obtained were stimulated with 50 ng/ml of PMA (Sigma), 500 ng/ml Ionomycin (Sigma) and 1/1000 of golgi stop (ebiosicence) during 3 h at 370 to analyze by flow cytometry the phenotype of tumor-infiltrated lymphocytes. By gating on CD45+CD4+ (A-B) or CD45+CD8+ T cells populations (C-D) as well as live/dead stain, the tumor-infiltrating (A) T regulatory cells (Treg; Foxp3+), (B) effector CD4+ cells (Th1; T-bet+) and (C) cytotoxic CD8 T cells (CTL; IFN-γ+Granzym B+) were determined. The dots in the graphs indicate the relative number of cell subpopulations (A) T reg, (B) Th1 and (C) CTL present into tumor. The vehicle condition was normalized to 1. (D) measure of the PD-1 expression on the surface of CTL cells infiltrated inside tumors. The dots in the graph represents the percentage of PD-1 negative CD8+ cells inside the tumors.

FIGS. 18A-B. The silencing of LXRβ in tumor cells modifies the effect of DDA treatment on the ratio of macrophage infiltrated inside tumor. Animals were grafted with tumor cells and treated as described in FIG. 2 and the suspensions of tumor cells were stained for macrophage phenotype. By gating on F4/80+CD11b+ cells, the percentage of macrophage (A) M1 (CD86+CD206−) and (B) M2 (CD86−CD206+) was determinated. The dots in the graphs represent the relative number of macrophage M1 and M2 infiltrated inside the tumors. The vehicle condition was normalized to 1.

FIGS. 19A-I. The silencing of LXRβ in tumor cells modifies the effect of DDA treatment on dendritic cells infiltrated inside tumor. Animals were grafted with tumor cells and treated as described in FIG. 2 and the suspensions of tumor cells were stained for dendritic cell phenotype. By gating on live cells, the relative number of (A) MDSC (CD11b+Ly6G+ Ly6Cint), (B) dendritic cells CD11c+ and (C) CD11c+CD8α+ (CD86−CD206+) was assessed. (D-E) The dots in the graphs represent the ratio between MDSC and either (D) dendritic cells CD11c+CD8α+ or (E) CD11c+. The vehicle was normalized to 1 and graphs are representative of three independent experiments. (E-H) The level of the migratory receptor CCR-7 (E, G) and the mature marker (F, H) expressed on the surface of dendritic cells CD11c+ (E-F) and CD11c+CD8α+ (G-H) were measured by flow cytometry and the mean of fluorescence (MFI) is indicated by bars in the graphs. MDSC: myeloid suppressive cells. CD11c+CD8α+: antigen presenting dendritic cells.

FIGS. 20A-C. The priming of T cells inside tumor side lymph node is dependent of the LXR expressed by tumor cells. Animals were grafted with tumor cells and treated as described in FIG. 2. At the end of the experiments, tumor side lymph nodes (mesenteric, auxiliary and brachial) were collected, dissociated and stimulated in vitro with 50 ng/ml of PMA (Sigma), 500 ng/ml Ionomycin (Sigma) and 1/1000 of golgi stop (ebiosicence) during 3 h at 370 to analyze by flow cytometry the phenotype of T cells. (A-C) The dots in the graphs represent the relative number of (A) T regulatory cells (CD4+ Foxp3+), (B) Th1 CD4+ cells (CD4+ T-bet+) and (C) cytotoxic CD8+ T cells (CD8+ Granzym B+ IFNγ+). The vehicle was normalized to 1. Graphs are representative of three independent experiments.

FIGS. 21A-E. DDA-exosome treatment controls tumor growth and animal survival. Immunocompetent C57BL/6 mice were implanted subcutaneously (s.c) with 300 000 E0771 (ER+) mouse mammary cancer cells expressing the LXRβ (E0 ShC, full symbol) or silenced for the LXRβ expression (E0 ShLXRβ, empty symbol). When the tumor reached a volume of 50-100 mm³, animals (n=8-10 mice per group) were treated at time indicated with either DDA at 0.37 μg/kg (Affichem) (▪,□) or vehicle (●,∘) once per day, every two, or with 5 μg exosomes purified from the media of cell treated with 2.5 μM DDA for 24 h (DDA-exo, ▾,∇) or with vehicle (C-exo ▴,Δ), or with a combo treatment DDA+DDA-exo ( [symbol] ) or DDA+C-exo (⋄,♦). Animals were monitored over time for tumor growth, the dots in the graphs show tumor volumes 20 days post-treatment. The tumor volume was determined by direct measurement with a caliper and was calculated using the formula (width²×length)/2. The mean tumour volume±s.e.m is shown. The Kaplan-Meier method was used to compare the mice survival. Data are representative of 2 experiments.

FIGS. 22A-B. DDA-exosome treatment protect against a rechallenge with tumor cells. The mice, grafted with E0771 shC or E0771 shLXRβ and treated as indicated, which exhibited complete tumor eradication from previous experiments (FIG. 7), were injected in the tail vein (i.v.) with 300 000 E0771 tumor cells. Since no mice treated with vehicle have survived from the experiments of FIG. 7, we have used as control mice, healthy mice that have not been injected previously with tumor cells. These control mice were injected in the tail vein with E0771 tumor cells (n=3), as the survival mice of the experiments of FIG. 7. Seven days later, all the mice were killed and their lungs were isolated and stained intratracheally with 15% India Black Ink solution and tumor surface (not stained with the black ink) relative to healthy surface of the lungs (stained with the black ink) was measured with the Image J software. The bar graphs represent the mean of tumor-free total lung surface (in %) from mice having been grafted subcutaneously with (A) E0711shC or (B) E0711 shLXRβ (except control mice), and rechallenged with E0771 tumor cells and treated as indicated. Data are representative of 2 experiments.

FIGS. 23A-C. The cytokine gradient modified by DDA and DDA-exosome treatment is dependent on the LXRβ expressed in the tumor cells. Measurement of cytokines by multiplex cytokine bead array (CBA) in plasma of mice grafted with E0771 ShC or E0711 ShLXRβ as previously described in FIG. 7. Seven days after different treatments, the blood was collected and the plasma were separated by high speed centrifugation. The results are expressed as pg/mL concentration, the bar grafts (A, B) show anti-tumor cytokines, (C) pro-tumor cytokine.

### EXAMPLE 1

Methods

Cell Culture. E0771, B16F10 and SKMEL28 tumor cells were from the American Type Culture Collection (ATCC, USA). Cells were grown at 37° C. in humidified atmosphere with 5% C02 in media containing 2 mM L-glutamine, 50 U/ml of penicillin/streptomycin and 10% fetal bovine serum (FBS) (for SKMEL-28, FBS was heated for 1 h at 56° C.). E0771 cell were cultured in RPMI 1640 medium supplemented 1% Hepes. B16F10 (passages did not exceed 20) were grown in DMEM 4 g/l sucrose plus 2 mM glutamine and SKMEL28 in RPMI 1640. The cells were splitted at 80% confluence.

Obtention of LXRβ knock-down cells. SKMEL28 cells (5×105) or E0771 (3×10⁶) were transfected with the Neon Transfection System (Invitrogen) with 1 μg or 3 μg of small hairpin RNA targeting human LXRβ or mouse LXRβ (two different shRNA were used) or with 1 μg control ShRNA. Transfected cells were selected in multiwell plates (10 000 cells/well) with puromycin ranging from 1-10 μg/ml. Two clones transfected with two different shRNA against LXRβ with LXRβ expression knocked-down by 70% and 80% (for SKMEL28) and by 90% and 95% (for E0771) and two control clones were selected.

Exosome preparation. Cells were seeded in complete medium at 50% confluence with exosome-free FBS, obtained after ultracentrifugion overnight at 110 000×g to eliminate serum exosomes and other microvesicles, and sterilized through a 0.2 μm filter. Human SKMEL28 cells were incubated with 2.5 μM DDA or vehicle (ethanol 1/1000 v/v final) for 24 h and mouse B16F10 cells were incubated with 1 μM DDA for 24 h. After this time, cell culture medium was collected and exosomes were purified from the cell culture medium by differential centrifugations. Briefly, cell culture medium was sequentially centrifuged at 4° C. at 1200×g for 5 min and 10 000×g for 30 min. Exosomes were then pelleted at 110 000×g for 70 min, resuspended in 5 ml PBS and centrifuged again at 110 000×g for 70 min. Final exosome pellet was diluted in PBS. For in vivo experiments, exosome were prepared in sterile conditions or sterilized by filtration through a 0.2 μm culture sterilization filter before injection into mice.

Exosome quantification. 1) Protein content in exosomes was quantified by the spectrophotometric method of Lowry in presence of 0.1% w/v sodium dodecyl phosphate. 2) Exosomes were also quantified by flow cytometry following labeling with the fluorescent lipid Bodipy-ceramide (Invitrogen-Molecular Probes) for 1 hour at 37° C. Excess of Bodipy-ceramide was removed by filtration and washing through the 1000 kDa Vivaspin filter and exosomes were quantitated by FACS. 3) Numeration of exosomes vesicles was performed either by nano tracking analysis (Nanosight equipment, Malvern, France) or by TRPS (tunable resistive pulse sensing) technology (qNano equipment, Izon, UK).

Exosome characterization. By flow cytometry: (tetraspanin analysis), exosomes (10 μg) were bound onto 10 μl of latex beads (Interfacial Dynamics/Invitrogen) in 200 μl PBS for 1 hour at 25° C. with gentle periodical shaking. Free sites on latex beads were saturated with 100 μl vesicle-free FBS for 30 min at 25° C. Beads with bound exosomes were centrifuged for 5 min at 5000 rpm, washed in 200 μl PBS, and diluted in 100 μl FACS buffer. Specific primary antibody or control isotype (1:50) were added and incubated at room temperature for 30 min. After centrifugation and washing, secondary antibody (1:100) was added and incubated for 30 min at room temperature. Beads with bound antibody-labelled exosomes were diluted in 1 ml FACS buffer and analyzed by flow cytometry (FACScalibur, Becton-Dickinson). By western-blot analysis: 5-20 μg exosomes were directly diluted in sample buffer and denaturated by heating at 60° C. for 10 min. Equal amounts of proteins were deposited in each well and proteins were resolved in SDS-PAGE and transferred onto PVDF membranes, saturated with 5% w/v non-fat milk in TBS-Tween 0.1%. Antibodies were added in 1% w/v non-fat milk in TBS-Tween 0.1% at the indicated dilutions according to the manufacturer. Revelation from immunoblotting was performed by enhanced chemiluminescence and analysed by ChemiDoc imager (BioRad) or by Pxi imager (Ozyme). By sucrose gradient: the density of exosomes was measured through a sucrose gradient. 50 μg exosomes in 100 μl PBS were deposited on top of a discontinuous gradient constituted by 9 layers of increasing sucrose concentration from 0.25 M to 2.25 M and a cushion of 2.5 M sucrose, and centrifugated at 160 000 g for 16 hours in swinging buckets. Fractions of 1 ml were harvested, diluted in 10 ml PBS and centrifugated for 2 h at 110 000×g. Pellets were recovered in Laemli buffer and their protein content resolved through SDS-PAGE, then probed for expression of CD9, Alix, Hsp70 and tyrosinase as indicated.

Prostaglandin determination. PGE2 in exosomes from SKMEL-28 was determined at the lipidomic facility of IMBL/INSA-Lyon from 70 μg protein. Briefly lipids were extracted with ethylacetate, samples were spiked with 10 ng of deuterated prostaglandins standards (Cayman), lipids separated by UHPLC and characterized by MS/MS. PGE2 in exosomes from B16F10 cells were determined from samples extracted by methanol/water, spiked with standards and analyzed by LC/ESI-MS.

Generation and treatment of DC: Peripheral blood mononuclear cells were isolated from human peripheral blood of healthy donors by standard density gradient centrifugation on Ficoll-Hypaque (GE Healthcare). Mononuclear cells were separated from peripheral blood lymphocytes (PBL) by centrifugation on a 50% Percoll solution (GE Healthcare). Monocytes were purified by immunomagnetic depletion (Life technologies, Rockville, Md., USA) using a cocktail of monoclonal antibodies (Ab) anti-CD19 (4G7 hybridoma), anti-CD3 (OKT3, ATCC, Rockville, Md., USA) and anti-CD56 (NKH1, Beckman Coulter, Fullerton, Calif., USA). Monocytes (purity>90%) were differentiated to immature DC (iDC) during 7 days with human rGM-CSF and rIL-4 (Human DC cytokine package, Peprotech) in RPMI 1640 supplemented with 2 mM glutamine, 10 mM Hepes, 40 ng/ml gentamycin (Life Technologies) and 10% FBS. Cells were treated at day 6 for 24 h with 20 μg exosomes. All cells and supernatants were collected at day 7. Control mature DC (mDC) were obtained by adding 1 μg/ml LPS (from Escherichia coli 0127:B8) at day 6 for 24 h. All DC were more than 95% pure as assessed by CD14 and CD1a labeling. DC Phenotyping: DC phenotype was analyzed on a FACSCanto (BD Biosciences, Le Pont de Claix, France) using FITC-conjugated anti-CD14, -HLA-DR, -CD80, -CD54, and PE-conjugated anti-CD1a, -CD86, -CD83 and -CD40 (Beckman Coulter). Mixed Lymphocyte Reaction (MLR): T lymphocytes were purified from PBL, after Ficoll-Hypaque and Percoll gradient centrifugation as described above, by immunomagnetic depletion using a cocktail of monoclonal Ab anti-CD19 (4G7), anti-CD56 (NKH1), anti-CD16 (3G8), anti-CD14 (RMO52) and anti-glycophorin A (11E4B7.6) (Beckman Coulter). T lymphocytes were more than 95% pure as assessed by CD3 labeling. Primary MLR were conducted in 96-well flat-bottom culture with various DC/T lymphocyte ratios (1/10; 1/20; 1/40).

Healthy C57BL/6 mice treatment with DDA: 6 weeks healthy C57BL/6 mice (from Janvier laboratory) were injected intraperitoneally (IP) with 100 μl of DDA (synthesized by Affichem) (0.37 μg/kg, 5 mg/kg or 20 mg/kg in sterile water) or with the solvent vehicle (control) every 5 days. Mice were killed at day 20 and single-cell suspension was prepared from spleen for flow cytometry analysis.

Tumor growth analysis. All of the animal procedures for the care and use of laboratory animals were conducted according to the guidelines of our institution and followed the general regulations governing animal experimentation. Exponentially growing cells were harvested, washed two times in PBS, and resuspended in PBS at the indicated concentrations. B16F10 tumors were obtained by subcutaneous transplantation of 35 000 cells in 150 μl into the flank of C57BL/6 or Balb/c female mice respectively. Then 1 μg of exosomes isolated from culture medium of cells treated with DDA or vehicle were injected once intra-dermally into the opposite flank. E0771, E0771 sh control and E0771 sh LXR tumors were prepared by subcutaneous transplantation of 300 000 cells in 100 μl PBS into the flank of C57B16 mice (6 week-old from Janvier laboratory). When the tumors reached a volume of 50 mm³ (around 10 days), the mice were injected intraperitoneally (IP) with 100 μl of DDA (0.37 μg/kg or 20 mg/kg in sterile water) or with the solvent vehicle (control). The treatment was repeated every 2 or 5 days as indicated until the end of experiment. The tumor volume was determined every 2-3 d by direct measurement with calipers and calculated using the formula [width²×length]/2. The Kaplan-Meier method was used to compare mice survival.

Tumor dissociation: Freshly excised tumors were trimmed of skin, fat, and necrotic tissue and minced in cold Hanks' medium. The minced tumor pieces were placed in an enzyme solution consisting of collagenase type D at 1 mg/ml and DNase type 1 at 20 μg/ml in Hanks' medium at 37° C. After 30 min of dissociation, the cell suspension was collected, washed with Hank's medium, and then suspended in PBS 1×, 0.5% BSA, 0.02% azide and 200 mM EDTA (Facs medium).

Analysis of immune cells by flow cytometry. Immune cells from the tumors were stained with the indicated fluorescent-labelled antibodies: anti mouse α-CD4, α-CD8, α-T-bet, α-Foxp3, α-granzyme B, α-PD-1, α-CD44, α-Ly6C, α-Ly6G, α-CD11b, α-CD11c, α-CD206, α-CD86, α-IL10, α-IL-6, α-IL-4 purchased from eBioscience or Biolegend. Intracellular staining for T-bet, Foxp3, IFNg, Granzyme B, IL-10, IL-4 and IL-6 was performed according the manufacturer's protocol from Biolegend. To observe the cytotoxic CD8 T cells, the tumor suspension was prealably stimulated 2 h in vitro with a cocktail of PMA (50 ng/ml), ionomycine (500 ng/ml) and golgistop (concentration from manufacture BD Pharmingen), then the cells were stained with specific antibodies. To set the gates, flow cytometry dot plots were based on comparison with isotype control. Flow cytometry measurements of single-cells suspension were performed on a Fortessa 20X (BD pharmingen) and data were analyzed using FlowJo software.

Cells isolation. Single-cells leukocyte suspensions were obtained from spleens of C57BL/6 mice. Naive CD4 or CD8 T cells are isolated by depletion of memory CD4 or CD8 T cells and non-CD4 or non-CD8 T cells according the manufacturer's protocol from Miltenyi kit (Miltenyi biotec). Purities of CD4+CD44l^{ow} CD62L^{high} or CD8+CD44l^{ow} CD62L^{high} T cells after isolation were >98%

Immune cell culture. Isolated CD4+ or CD8+ T cells were cultures in 96-well flat bottom plates (0.25×10⁶ cells per wells) in 0.25 ml of complete RPMI 1640 media (10% FBS, 1% penicillin/Streptomycin, 1% sodium pyruvate, 1% HEPES and 50 μM b-mercaptoethanol) in the presence of 10 μg/ml plate-bound anti-mouse CD3 (2C11) and 2 μg/ml soluble α-CD28 (LEAF) in addition to 50 ng/ml of recombinant IL-2 (e-bioscience). DDA (synthesized by Affichem) diluted in the solvent vehicle was added at increasing concentration (0-1-10-100 and 1000 nM). Cells were cultured in polarizing Th1 (20 ng/ml of recombinant IL-2 and 10 μg/ml of anti-IL4), Th2 (50 ng/ml of recombinant IL-4, 10 μg/ml of anti-IFNg), Th17 (10 ng/ml of recombinant TGF-b, 100 ng/ml of recombinant IL-6, 10 μg/ml of anti-IFN-g and 10 μg/ml of anti-IL4) or Treg (10 ng/ml of recombinant TGF-b, 10 μg/ml of anti-IFNg and 10 μg/ml of anti-IL4) conditions. All recombinant cytokines were purchased from Peprotech and antibodies were purchased from eBioscience. After 5 days of culture, cells were collected and analyzed by flow cytometry. To investigate the impact of DDA on polarization of CD4 or CD8 naive T cells, DDA or the solvent vehicle was added at the beginning of culture at Day 0 or at Day 4 and cells was analyzed at day 5 by flow cytometry.

Statistical analyzes. Tumor growth curves in animals were analyzed for significance by the analysis of variance (ANOVA). In other experiments, significant differences in the quantitative data between the control and the treated group were analysed using the Student's t-test for unpaired variables (Graph Pad Prism software). In all figures, *, ** and *** refer to P<0.05, P<0.01 and P<0.001 compared with the control (vehicle), unless otherwise specified.

Results

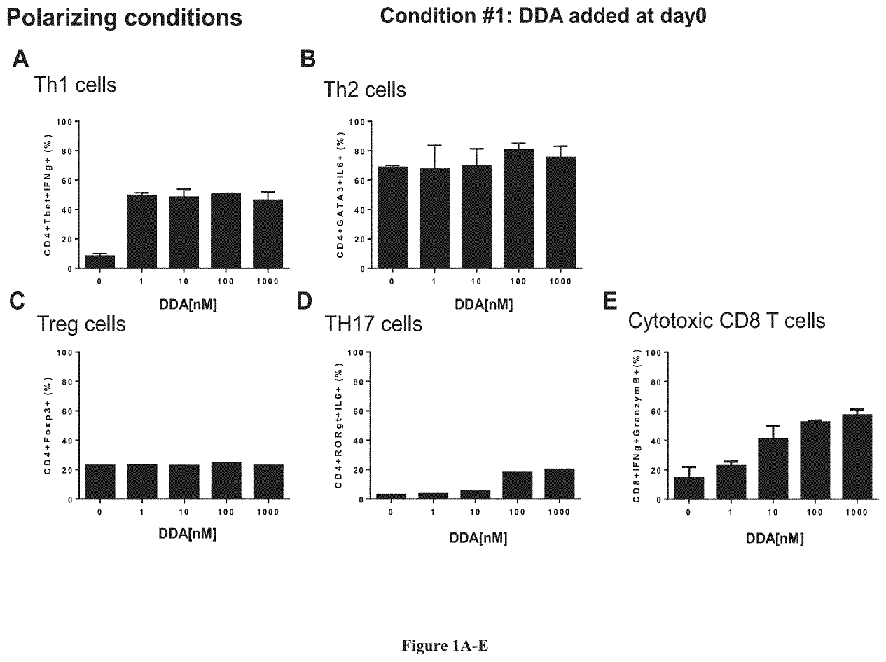

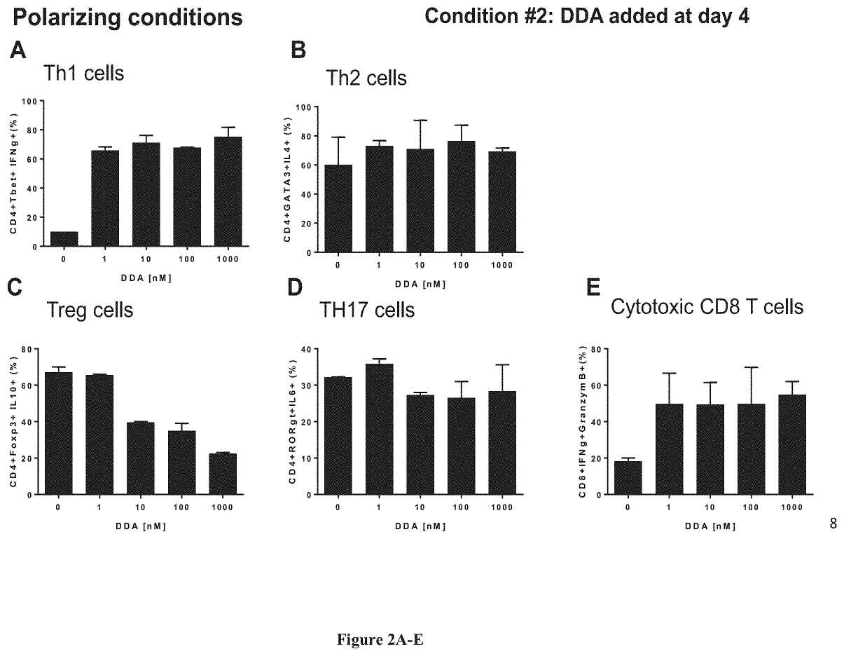

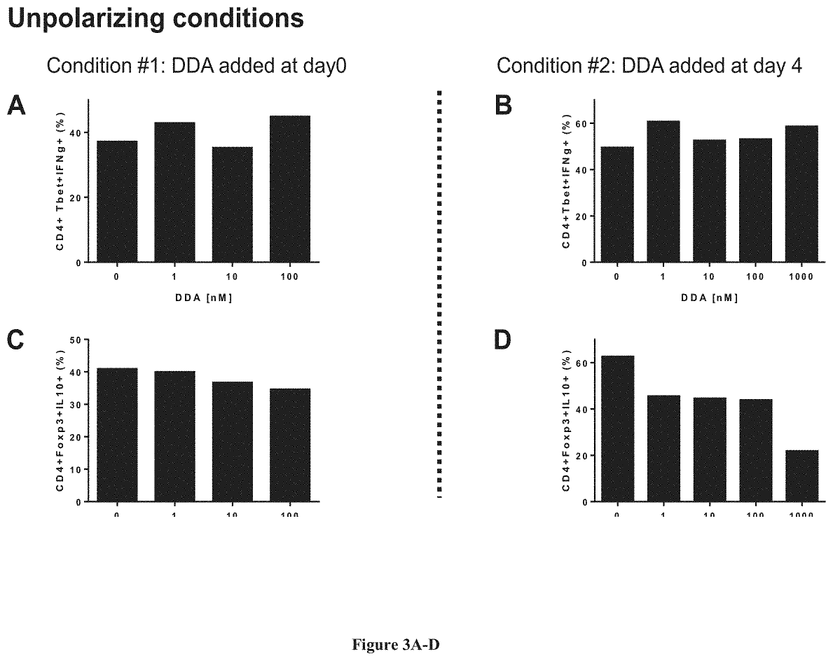

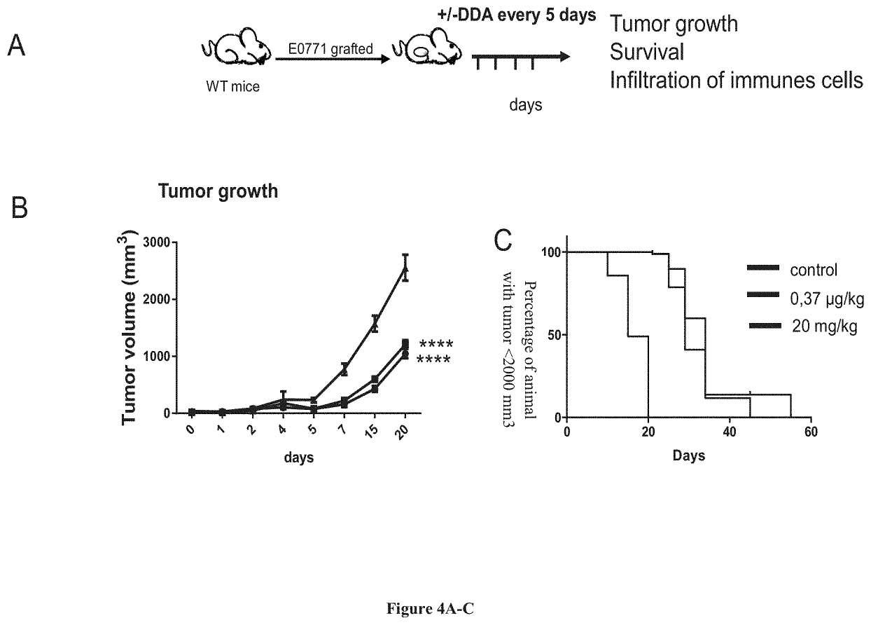

Results depicted in FIG. 1 show clearly that DDA increases the differentiation of Th0 into Th1 from 1 nM concentrations, the differentiation of Th0 into Th17 from 100 nM and the differentiation of naïve CD8 T cells into functional cytotoxic CD8 T cells from 10 nM. In contrast, DDA treatment has no effect on Th2 and Treg differentiation. When DDA was added at day 4, the differentiation of Th0 into Th1 and naïve CD8 T cells into functional cytotoxic CD8 T cells is also increased from 1 nM. In addition, DDA has no effect on the differentiation into Th17 and Th2. Importantly, DDA inhibits the differentiation of Th0 into Treg (FIG. 2). The results depicted in FIG. 3 show that DDA does not activated Th0 into Th1 differentiation but inhibits the differentiation of Th0 into Treg phenotype (more impressive in condition #2, Day 4).

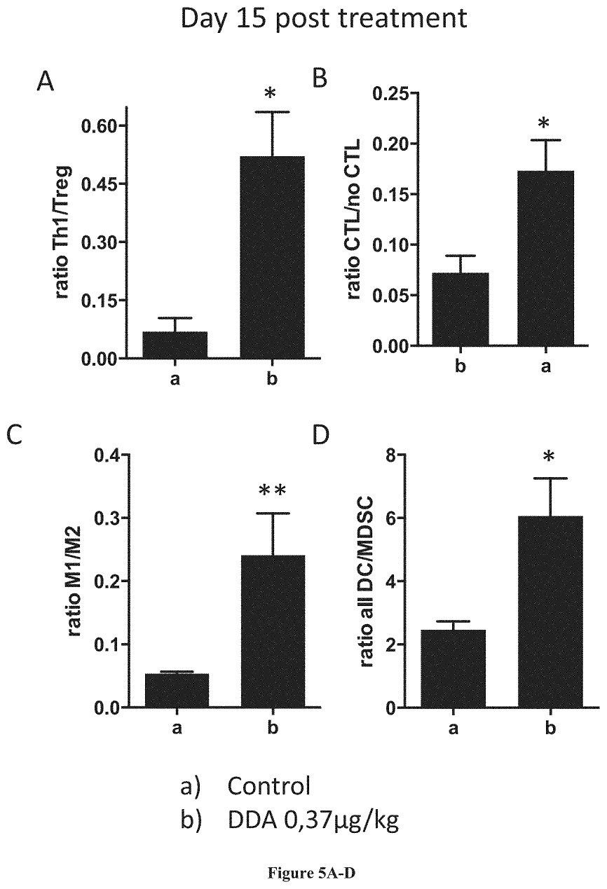

Whatever DDA concentrations, DDA treatment inhibits tumor growth and increases mice survival (FIG. 4). DDA treatment increases the infiltration of CD4 Th1 cells, activated CD8 (CTL), dendritic cells (DC: CD11c+), and macrophage type M1 inside the tumor. Inversely, DDA treatment decreases the infiltration into the tumor of the regulatory CD4 Treg cells (Treg), inactivated CD8 (No CTL), myeloid derived suppressive cells (MDSC) and macrophage type M2 (FIG. 5). Collectively these data show that DDA treatment allows activation of the immune system against the tumor resulting in the control of tumor growth. The CD4 T cells acquire an activated phenotype which is underlined by the upregulation of CD44 at their surface at day 4 (data not shown). Inversely, DDA has no significant effect on CD8 T cells phenotype (data not shown).

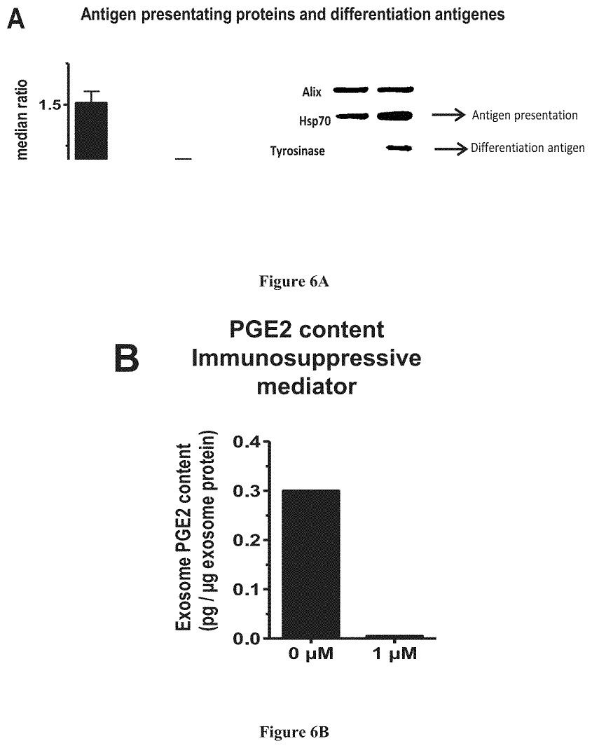

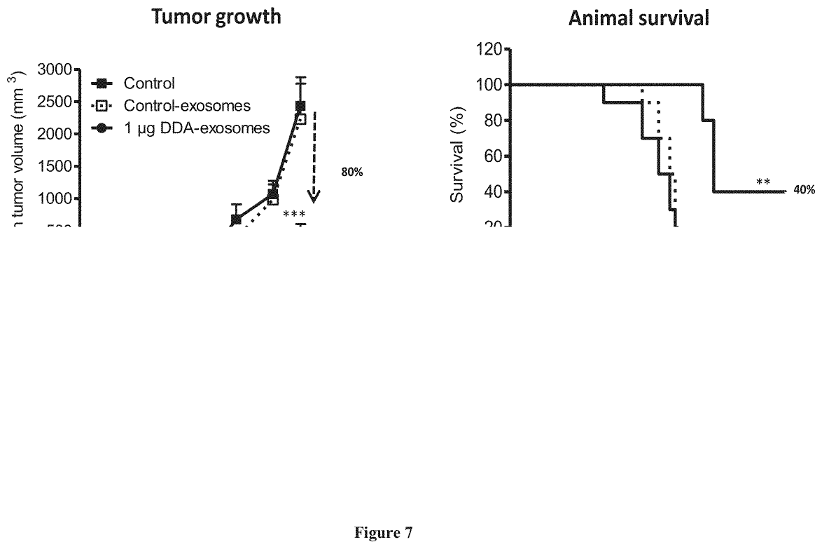

We show that DDA stimulates the amount of multivesicular bodies (MVB) which contain the exosomes in B16F10 cells, observed by electronic microscopy. The vesicles purified from B16F10 cell culture media after treatment with 1 μM DDA for 24 h or the solvent vehicle were characterized as being exosomes considering their size analysed by electronic microscopy, their density and the presence of specific markers of exosomes such as CD9, CD81 and Lamp2 (data not shown). DDA stimulates the production of exosome secreted into the media by 1.5 to 2-fold in B16F10 cells (data not shown). This effect was also observed in human and murine mammary tumor cells (data not shown). Exosomes modified by DDA (DDA-exosomes) display a differentiated and immunogenic phenotype compared with control-exosomes (FIG. 6). More particularly a single injection of DDA-exosomes controls tumor growth and increases mice survival (FIG. 7). We performed similar experiments with SKMEL-28 cells and we demonstrated that DDA stimulates exosome secretion from human melanoma cells (data not shown). DDA-exosomes from human SKMEL-28 melanoma cells display a differentiated and immunogenic phenotype compared with control-exosomes (data not shown).

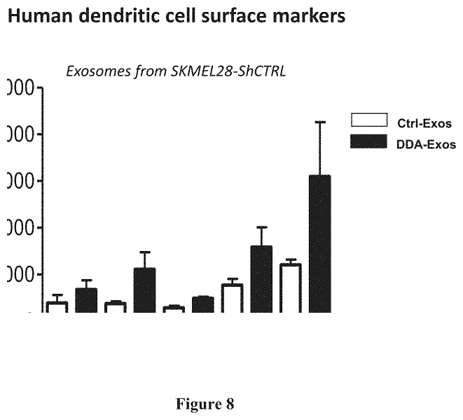

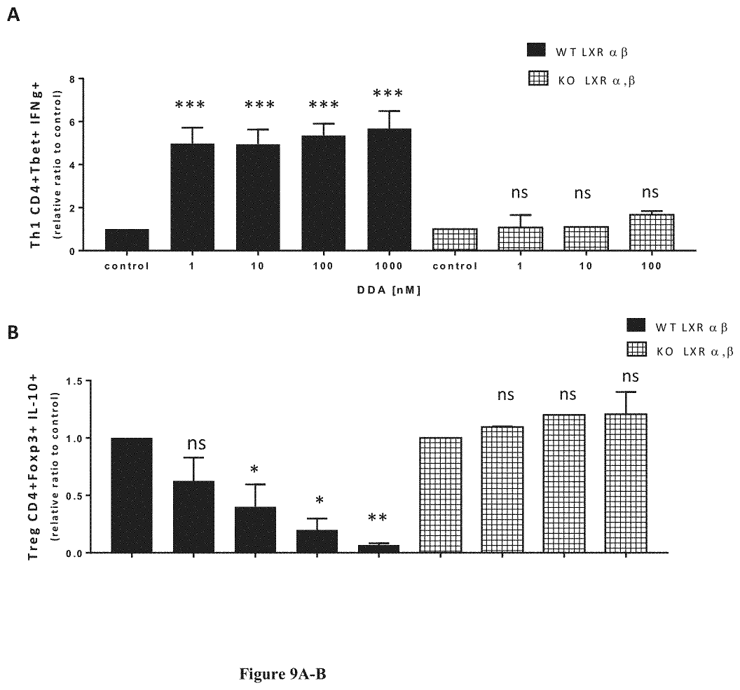

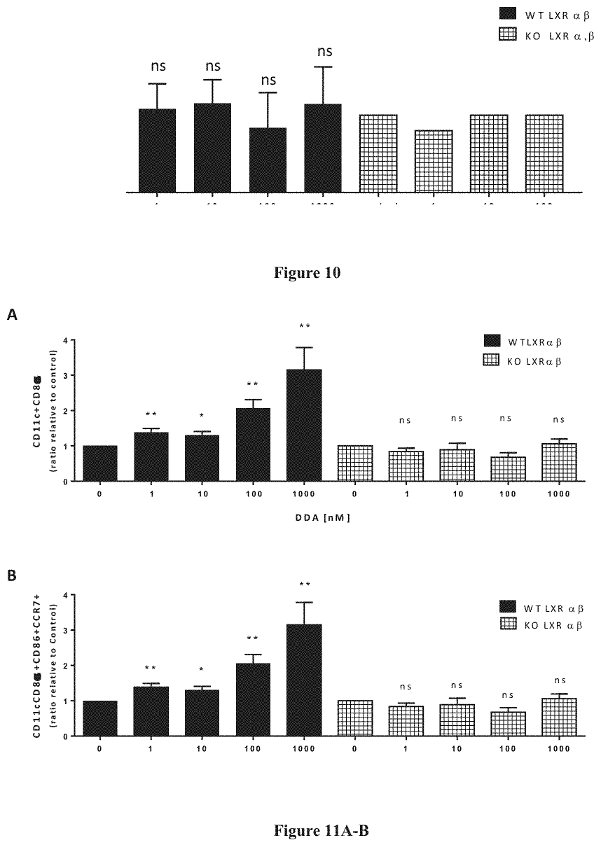

We then determined whether the liver X receptors (LXR), the receptors of DDA which are known to modulate of the immune system, were involved in the secretion and the phenotypic modification of DDA-exosomes in SKMEL-28 cells. The LXRbeta is the only subtype expressed in these cell type. We knocked-down the expression of the LXRbeta in SKMEL-28 by using specific shRNA against the LXRbeta (SKMEL-28-shLXRbeta) compared with control sh (SKMEL-28-shCTRL). SKMEL-28-shLXRbeta and SKMEL-28-shCTR cells were stimulated with 2.5 μM DDA for 24 h or with the solvent vehicle. Then, the exosomes were purified from the cell media, quantified and analysed. DDA (2.5 μM for 24 h) significantly increases the production of exosomes from SKMEL-28-shCTRL cells by about 2-fold while DDA does not stimulate the production of exosomes from SKMEL-28-shLXRbeta, indicating that LXRbeta mediates DDA-induced exosome secretion. DDA produces exosomes from SKMEL-28-shCTRL cells enriched in molecules involved in MVB trafficking (rab27a and b), antigen presentation (HSP70), antigen of differentiation (Melan A, tyrosinase, TRP2) and DC «eat-me» signal (calreticuline). In contrast, DDA produces exosomes from SKMEL-28-shLXRbeta cells that are not enriched in molecules involved in MVB trafficking (rab27a and b), antigen presentation (HSP70), antigen of differentiation (Melan A, tyrosinase, TRP2) and DC «eat-me» signal (calreticuline). These data indicate that the LXRbeta mediates DDA-induced the phenotypic modification of exosome. To determine the immunogenic properties of DDA-exosomes and the implication of LXRbeta in these effects we studied the impact of DDA-exosomes purified from SKMEL-28-shCTRL or SKMEL-28-shLXRbeta cells on dendritic cell maturation. DDA-exosomes purified from human SKMEL-28-shCTRL cells media increase cell surface markers of mature human dendritic cells (FIG. 8). DDA-exosomes purified from SKMEL-28-shCTRL cells media stimulate the secretion of immunoactivating cytokines which are secreted by mature dendritic cells. The IL12/IL10 ratio is strongly increased. This effect is not observed with DDA-exosomes purified from SKMEL-28-shLXRbeta cells media indicating that DC maturation by DDA-exosomes is dependent on the expression LXRbeta in the parental cells (data non shown). Dendritic cells maturated by DDA-exosomes purified from the media of SKMEL-28-shCTRL cells stimulate naive T lymphocytes to produce interferon gamma indicating that DDA-exosomes activate the functionality of naive T lymphocytes toward a immunostimulator Th1 phenotype (INFg production>>IL13, IL6 production). These effects are abolished when similar experiments were realized with DDA-exosomes purified from the media of SKMEL-28-shLXRbeta cells (data non shown). These data indicate that the effect of DDA-exosomes on DC-functionality depends on LXRbeta expression in the parental cancer cells. In conclusion, LXRb expressed in cancer cells drives the effect of DDA on exosome secretion, phenotype modification and immunogenicity.

### EXAMPLE 2

FIG. 9 shows that DDA increases the differentiation of Th0 into Th1 and decreases the differentiation of Th0 into Treg. Moreover, the effect is dependent of LXR expression, because on its absence the DDA effect on Th1 and Treg differentiation is abrogated.

FIG. 10 shows that that DDA has no impact on Th2 differentiation and is independent of LXR.

FIG. 11 shows that DDA increases the differentiation of CD11c into CD11c CD8a+ and their maturation, and that this effect is dependent of LXR expression since it is abolished in absence of LXR.

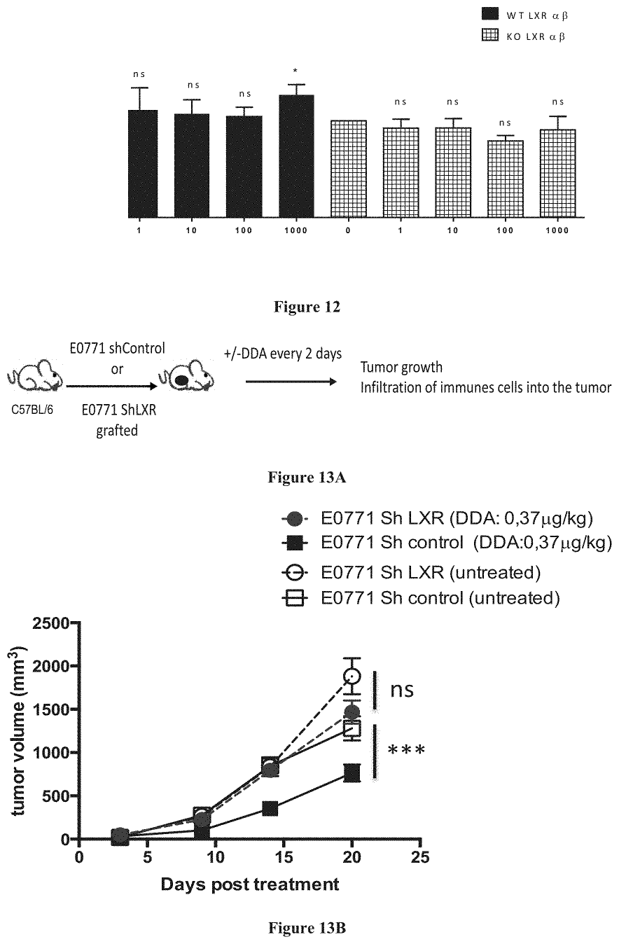

FIG. 12 shows that 1 μM DDA increases MHC II expression at the surface of CD11c dendritic cells and this effect is dependent of the expression of the LXR since it is abrogated in absence of LXR.

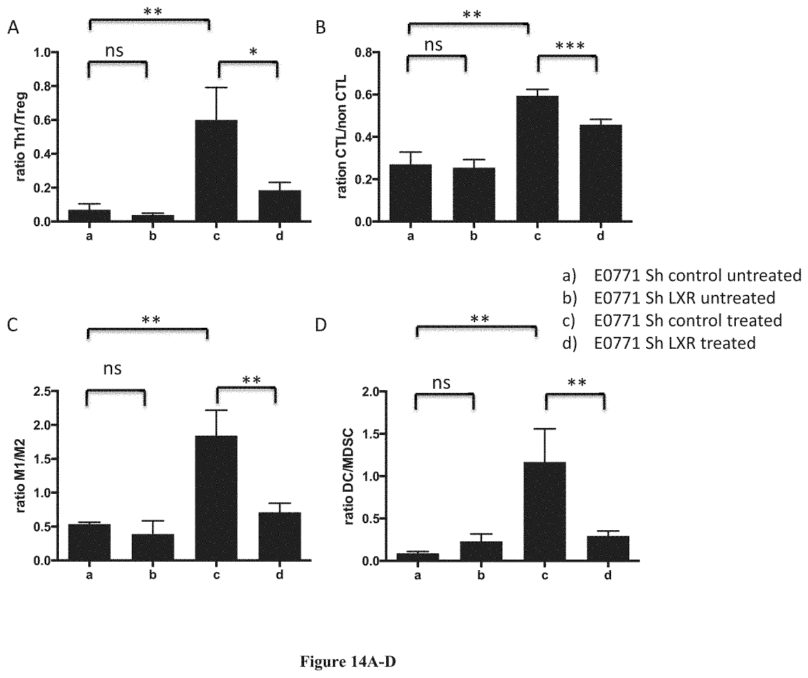

FIG. 13 shows that DDA significantly controls the growth of tumors expressing the LXRβ (E0771 sh control) while this effect is abolished in tumors knocked down for the expression of the LXRβ (E0771 sh LXR), indicating that the LXRβ mediates the control of tumor growth by DDA.

FIG. 14 shows that the activation of an immuno-active microenvironment inside the tumors under DDA treatment is dependent of the expression of the LXRβ in the tumors.

### EXAMPLE 3

Material and Methods

Exosome preparation. Mouse mammary E0771 cells (ATCC) were seeded in DMEM with 10% exosome-free FBS at 50% confluence. Exosome-free FBS were obtained after ultracentrifugion overnight at 110 000×g to eliminate serum exosomes and other microvesicles, and sterilized through a 0.2 μm filter. E0711 cells were incubated with 1.5 μM DDA or vehicle (ethanol 1/1000 v/v final) for 24 h. After this time, cell culture medium was collected and exosomes from cells treated with DDA (DDA-exo) or with the vehicle (C-exo) were purified from the cell culture medium by differential centrifugations. Briefly, cell culture medium was sequentially centrifuged at 4° C. at 1200×g for 5 min and 10 000×g for 30 min. Exosomes were then pelleted at 110 000×g for 70 min, resuspended in 5 ml PBS and centrifuged again at 110 000×g for 70 min. Final exosome pellet was diluted in PBS. For in vivo experiments, exosomes were prepared in sterile conditions.

Animal experiments. All of the animal procedures for the care and use of laboratory animals were conducted according to the guidelines of our institution and followed the general regulations governing animal experimentation. E0771 exponentially growing cells were harvested, washed two times in PBS, and resuspended in PBS at the indicated concentrations. E0771 Shcontrol (shC) or E0711 ShLXRβ (shLXRβ) tumors were prepared by subcutaneous transplantation of 300 000 cells in 100 μl PBS into the flank of C57B16 mice (6 week-old from Janvier laboratory). When the tumors reached a volume of 50 mm3 (around 10 days), the mice were injected intraperitoneally (IP) with 100 μl of DDA (0.37 μg/in sterile water) or with the solvent vehicle (control) once per day and every two days. Depending of the experiments, the mice were also treated subcutaneously with 5 ug exosomes from E0711 tumor cells treated or not with DDA (DDA-exo versus C-exo) as described above or with 5 ug exosomes (DDA-exo versus C-exo) in combination with DDA (0.37 ug/kg). For the latter, the exosomes were injected 24 h after the first DDA treatment, then DDA treatment was maintained every two days once a day. The tumor volume was determined every 2-3 d by direct measurement with calipers and calculated using the formula [width2×length]/2. The Kaplan-Meier method was used to compare mice survival.

Organ dissociation and flow cytometry. The tumor-side lymph nodes were dissociated manually while for the tumor, gentlemac technology (Miltenyi) was used. Then, the suspension of tumor cells or lymph node were stimulated with 50 ng/ml of PMA (Sigma), 500 ng/ml Ionomycin (Sigma) and 1/1000 of golgi stop (ebiosicence) during 3 h at 37° C. After that, the single cell suspension were stained with the indicated fluorescent-labelled antibodies: anti mouse α-CD4, α-CD8, α-T-bet, α-Foxp3, α-granzyme B, α-PD-1, α-CD44, α-Ly6C, α-Ly6G, α-CD11b, α-CD11c, α-CD206 IL10, α-CD86 as well as live/dead stain purchased from eBioscience or Biolegend. Intracellular staining for T-bet, Foxp3, and Granzyme B, was performed according the manufacturer's protocol from Biolegend. To set the gates, flow cytometry dot plots were based on comparison with isotype control. Flow cytometry measurements of single-cells suspension were performed on a Fortessa 20X (BD pharmingen) and data were analyzed using FlowJo software.

Tumor rechallenge: Mice exhaling a complete eradication of E0771 shcontrol (shC) or E0771 Sh LXRβ (shLXRβ) tumors following treatment with DDA combined or not with control-exosomes or DDA-exosomes, were rechallenged with 300 000 E0771 cells injected into the tail vein of mice. Seven days later, their lungs were isolated and stained intratracheally with 15% India Black Ink solution and fixated in Fekete's solution (100 mL of 70% alcohol, 10 mL formalin, and 5 mL glacial acetic acid). The percentage of lung surface invaded by metastatic nodules was analyzed using NIH Image J software. Briefly, lung photographs were converted in gray scale; metastatic nodules (white staining) and healthy lung tissue (black staining) were defined using the threshold color parameter and the respective area measured.

Measurement of cytokine in plasma: Cytokine plasma levels were determined using commercially available kits, Cytometric Beads Array—CBA (BD Biosciences Pharmingen, USA) to quantify IFN-γ, IL-12 and RANTES. The CBA immunoassay was carried out according to the manufacturer instructions. Flow cytometry measurements were performed on a LSR II (BD pharmingen) and data were analyzed using FCAP array software (BD pharmingen).

Results

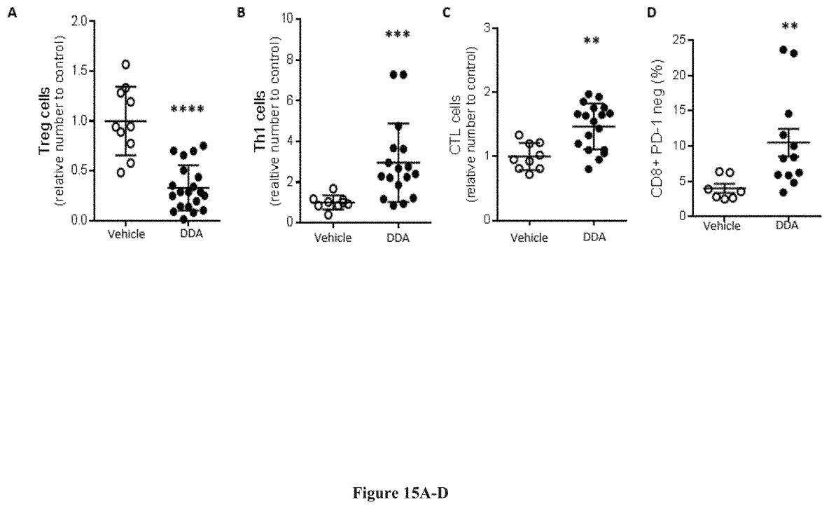

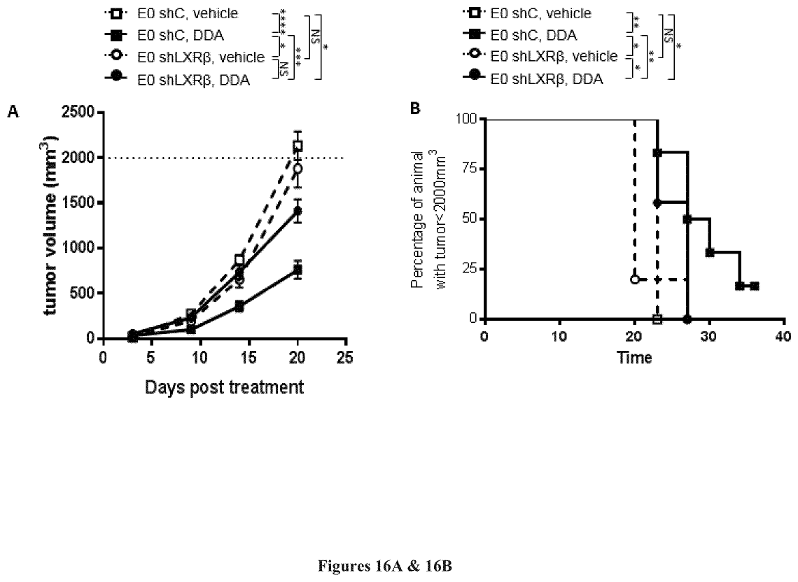

FIG. 15 shows that DDA decreases the number of T regulatory CD4 T cells and increases the activated CD4+ and CD8+ T cells inside tumors.

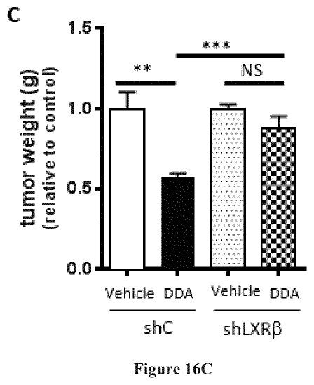

FIG. 16 shows that DDA inhibits tumor growth and increases animal survival by acting through the LXRβ expressed in tumor cells.

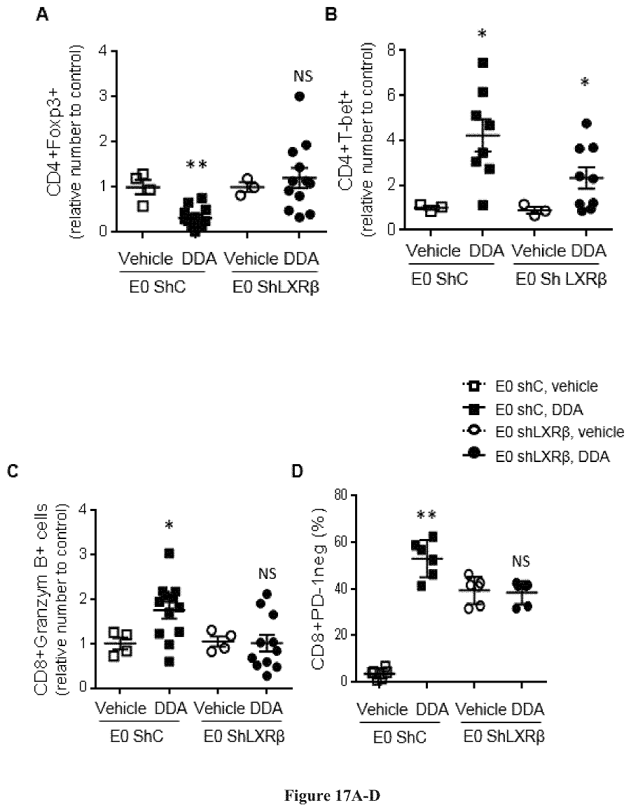

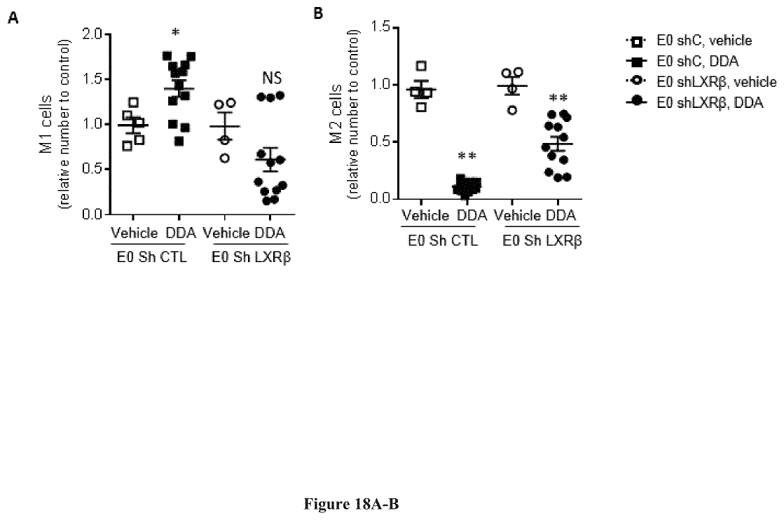

FIG. 17 shows that DDA decreases the number of T regulatory cells and increases the number of effector Th1CD4+ cells infiltrated into the tumors and increases the number of activated cytotoxic CD8+ T cells infiltrated into the tumors. The decrease expression of LXRB into the tumors abolished the effect of DDA on T regulatory and activated cytotoxic CD8+ T cells infiltrated into the tumors but had no effect on the effector Th1CD4+.

FIG. 18 shows that DDA increases the number of macrophages M1 infiltrated into the tumors and this effect is dependent of the LXRβ expressed in the tumors. DDA decreases the number of macrophages M2 infiltrated into the tumors. This effect is independent of the LXRβ expressed in the tumors.

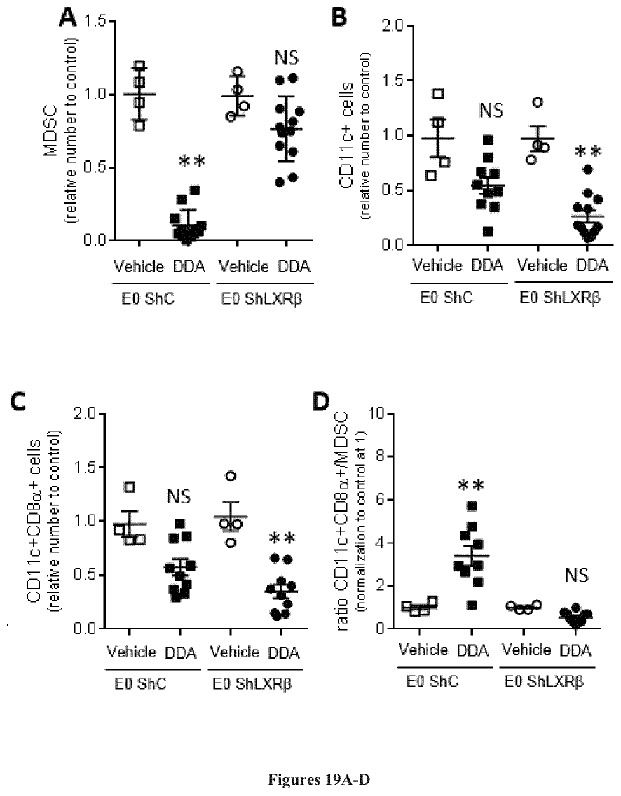

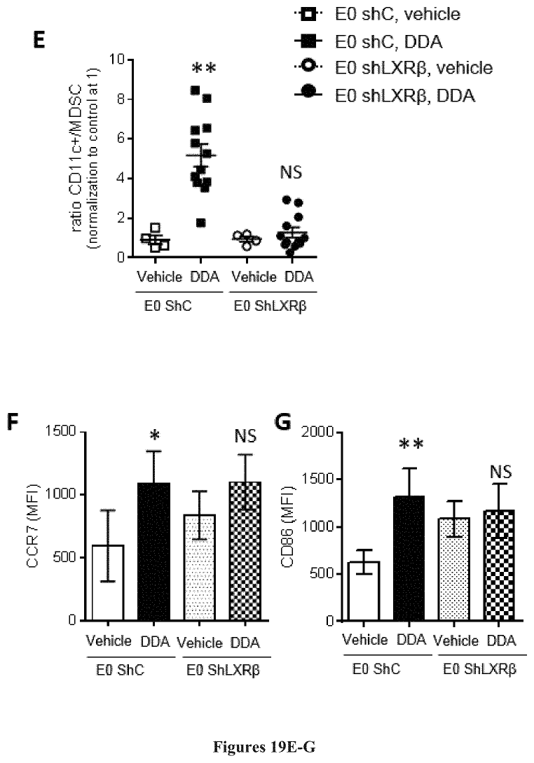

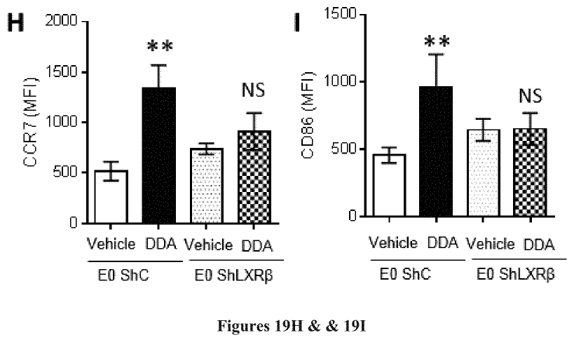

FIG. 19 shows that DDA decreases the number of MDSC infiltrated into the tumor and increases the ratio of dendritic cells CD11+ and CD11+ CD8α+ versus MDSC. These effects are abolished in tumors knocked-down for the LXRβ.

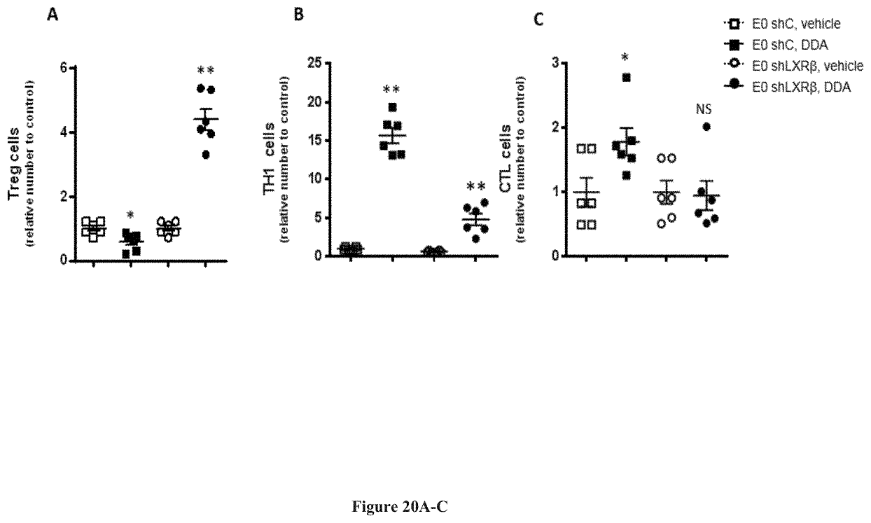

FIG. 20 shows that DDA decreases the number of Treg cells and increases the number of Th1CD4+ cells and cytotoxic CD8 T cells infiltrated into tumor side lymph nodes. The priming of T cells inside tumor side lymph nodes is dependent of the LXRβ expressed by tumor cells.

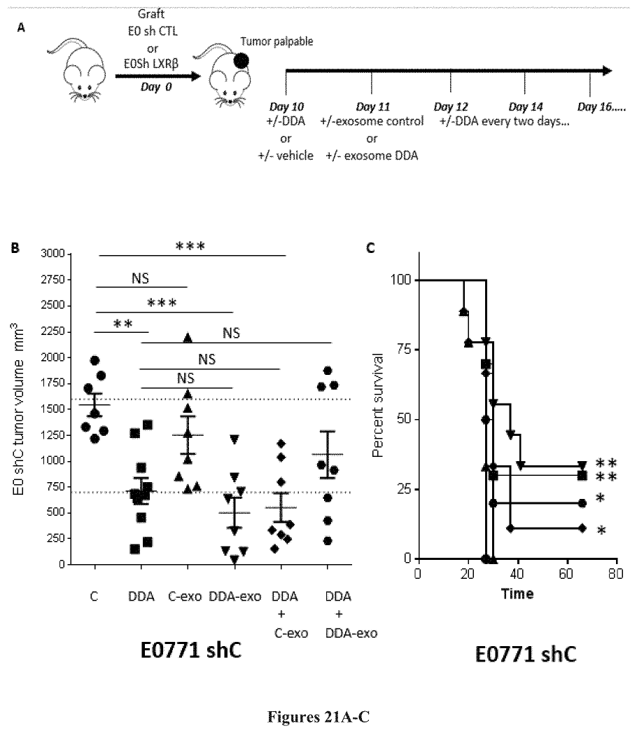

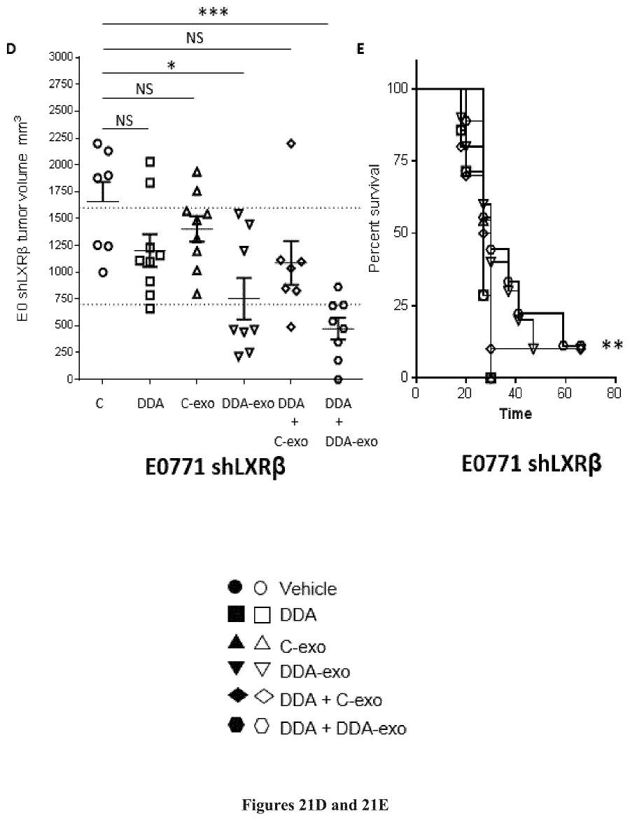

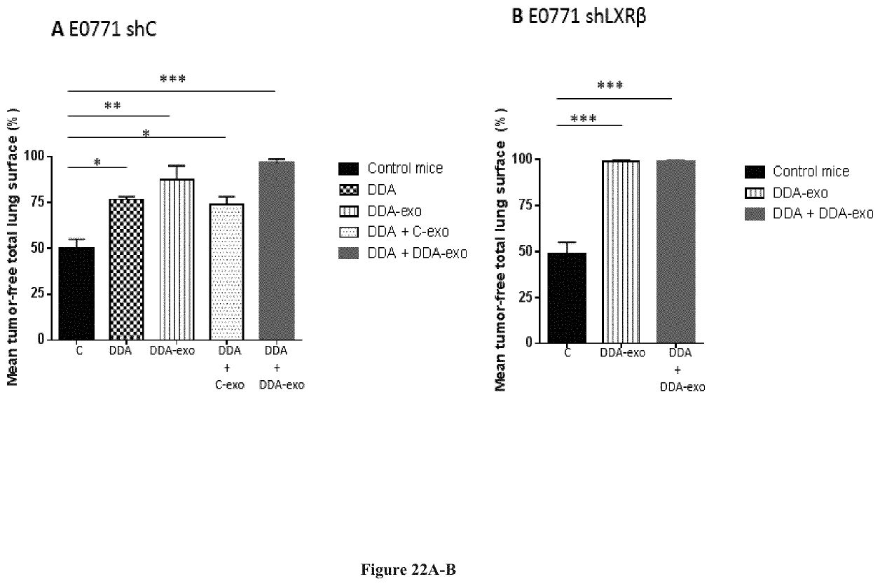

FIG. 21 shows that DDA-exosome treatment significantly decreases tumor growth and increases animal survival. Treatment with DDA-exosomes compensates the silencing of LXRβ on tumor cells and the loss of DDA response and increases animal survival and tumor-free mice.

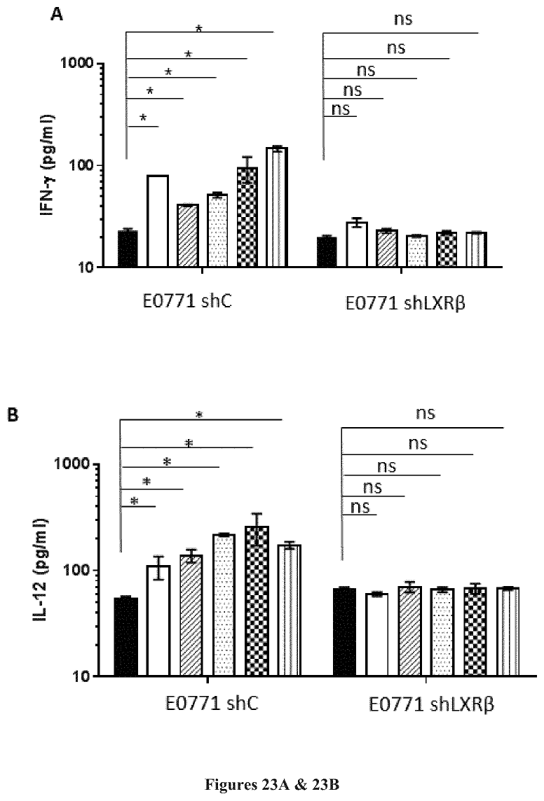

FIG. 22 shows that DDA-exosomes protect against a rechallenge with tumor cells expressing or not the LXRβ.

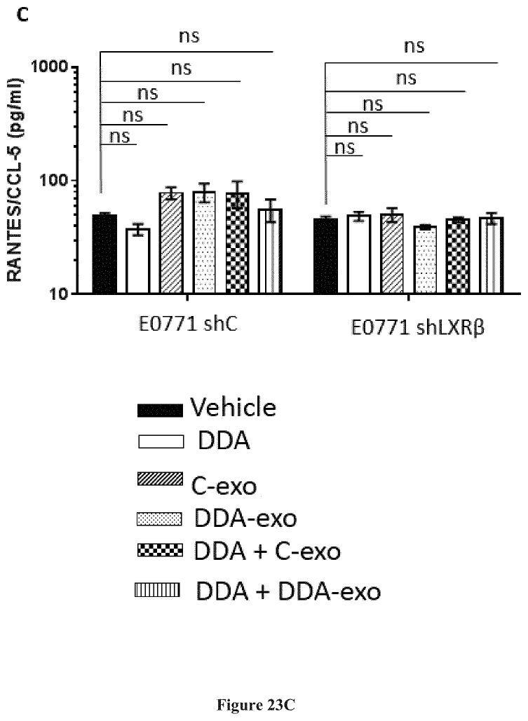

FIG. 23 shows that DDA-exosome treatment increases the anti-tumor cytokines, IFNγ and IL-12, in the blood of mice grafted with tumor expressing the LXRβ. These effects are abolished when animals were grafted with tumor silenced for the LXRβ. No increase was observed for the pro-tumor cytokine Rantes.

### REFERENCES

Throughout this application, various references describe the state of the art to which this invention pertains. The disclosures of these references are hereby incorporated by reference into the present disclosure.

1. A method of treating cancer in a subject in need thereof comprising administering a therapeutically effective amount of a population of DDA-exosomes, wherein the population of DDA-exosomes is obtained by a method comprising the steps of contacting a population of tumor cells with an amount of DDA for a time sufficient to induce exosomes releasing by the population of tumor cells.

2. The method according to claim 1, wherein the subject is administered with a vaccine composition comprising the population of DDA-exosomes, as an immunoadjuvant, together with one or more antigens for inducing an immune response against said one or more antigens.

3. The method according to claim 2, wherein the one or more antigens of the vaccine composition is a tumor associated antigen.

4. The method according to claim 1, wherein the subject is administered with a composition enriched by the population of DDA-exosomes.

5. The method according to claim 4, wherein the enriched composition comprises at least 10% of the population of DDA-exosomes.

6. The method according to claim 1, wherein the exosomes are tumor exosomes.

7. A method of treating cancer in a subject in need thereof comprising i) determining the presence or absence of LXRβ in a tumor tissue sample obtained from the subject and ii) administering to the subject a therapeutically effective amount of a population of DDA-exosomes when LXRβ is absent in the tumor tissue sample, wherein the population of DDA-exosomes is obtained by a method comprising the steps of contacting a population of tumor cells with an amount of DDA for a time sufficient to induce exosomes releasing by the population of tumor cells.

8. The method according to claim 7, wherein the population of DDA-exosomes is administered when LXRβ is present in the tumor tissue sample.

9. The method according to claim 7, wherein the subject is administered with a vaccine composition comprising the population of DDA-exosomes, as an immunoadjuvant, together with one or more antigens for inducing an immune response against said one or more antigens.

10. The method according to claim 9, wherein the one or more antigens of the vaccine composition is a tumor associated antigen.

11. The method according to claim 7, wherein the subject is administered with a composition enriched by the population of DDA-exosomes.

12. The method according to claim 11, wherein the enriched composition comprises at least 10% of the population of DDA-exosomes.

13. The method according to claim 7, wherein the exosomes are tumor exosomes.
